(19) United States
(12) Patent Application Publication
(10) Pub. No.: US 2023/0049294 A1
(43) Pub. Date: Feb. 16, 2023

(54) STEREOSELECTIVE PROCESS OF MANUFACTURE OF PURINE PHOSPHORAMIDATES

(71) Applicant: Atea Pharmaceuticals, Inc., Boston, MA (US)

(72) Inventor: Adel Moussa, Burlington, MA (US)

(73) Assignee: Atea Pharmaceuticals, Inc., Boston, MA (US)

(21) Appl. No.: 17/971,318

(22) Filed: Oct. 21, 2022

Related U.S. Application Data
(63) Continuation of application No. PCT/US2021/028416, filed on Apr. 21, 2021.
(60) Provisional application No. 63/014,561, filed on Apr. 23, 2020.
(60) Provisional application No. 63/015,331, filed on Apr. 24, 2020.

(51) Int. Cl. C07D 487/04

(52) U.S. Cl. CPC C07D 487/04

(57) ABSTRACT

The present invention provides stereoselective processes of manufacture for the phosphoramidate nucleotide Compound 1 or a pharmaceutically acceptable salt thereof.

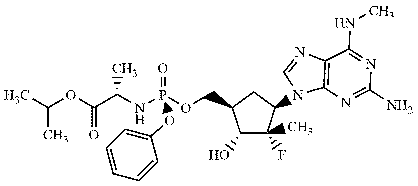 [SMILES: CNc1nc(N)nc2c1ncn2[C@@H]1C[C@H](CO[P@@](=O)(N[C@@H](C)C(=O)OC(C)C)Oc2ccccc2)[C@@H](O)[C@@]1(C)F]

### CROSS-REFERENCE TO RELATED APPLICATIONS

This application is a continuation of International Patent Application No. PCT/US2021/028416, filed in the U.S. Receiving Office on Apr. 21, 2021, which claims the benefit of U.S. Provisional Application No. 63/015,331, filed Apr. 24, 2020, and U.S. Provisional Application No. 63/014,561, filed Apr. 23, 2020. The entirety of each of these applications is incorporated herein by reference.

### FIELD OF THE INVENTION

The present invention provides stereoselective processes for the synthesis of purine phosphoramidate nucleotides and intermediates for the production thereof.

### BACKGROUND OF THE INVENTION

Nucleoside analogs have been developed as effective therapeutics for a number of diseases, including cancer, hepatitis C (HCV), hepatitis B (HBV), HIV, and human cytomegalovirus (HCMV). Nucleoside analogs have also been explored for RNA viral infections including viruses of the Flaviviridae family (Dengue Fever, Yellow Fever, Zika Virus), the Filoviridae family (Ebola Virus, Marburg virus), and the Coronaviridae family (SARS (severe acute respiratory syndrome) and MERS (Middle East respiratory syndrome coronavirus)).

To exert a therapeutic effect, a nucleoside analog must be converted to its respective 5’-O-triphosphate nucleotide. This activation takes place inside the cell via nucleoside and nucleotide kinases that convert the compound in a step-wise manner to the mono-, di-, and then triphosphorylated nucleotide. A rate-limiting step of this metabolism is often the initial phosphorylation to the 5’-O-monophosphate. For this reason, prodrugs of monophosphate nucleotide analogs, including phosphoramidate prodrugs (known as “ProTide” prodrugs) have been developed in an effort to bypass this first phosphorylation step. These nucleotide ProTide prodrugs were developed by Prof. McGuigan, for example as described in PCT Applications WO90/05736; WO90/10012; WO9629336; WO2000/047591; WO2001/083501; WO2001085749; WO2003/061670; and WO2005/012327. This synthetic technique may result in a mixture of nucleoside diastereomers where the stereochemistry is not defined at the phosphorus.

The phosphorus atom in a phosphoramidate is chiral and can exist in an Sₚ-configuration or an Rₚ-configuration. For many phosphoramidate nucleotides, one diastereomer, such as the Sₚ isomer, is more active than the Rₚ isomer and for that reason, it is advantageous to develop processes that either selectively afford the Sₚ- or Rₚ-isomer or include a step of separating the isomers. U.S. Pat Nos. 8,618,076; 8,642,756; and, 8,563,530 and PCT Applications WO 2011/123668; WO 2010/135569; and WO 2011/123645 assigned to Gilead/Pharmasset describe stereoselective processes for producing Sₚ- and Rₚ-phosphoramidate nucleotides. Other patents that describe processes for separating Sₚ-phosphoramidate diastereomers and stereoselective process for producing Sₚ-isomers include U.S. Pat 10,774,104 assigned to NuCana plc and U.S. Pat. Nos. 10,538,541; 10,689,413; and, 10,913,756 assigned to BrightGene Bio-Medical Technology.

Additional techniques for the stereoselective synthesis of phosphoramidate nucleotides include the use of chiral catalysts developed by Merck disclosed in U.S. Pat. Nos. 10,214,554 and 10,597,422 and DiRocco et al. (Science 2017, 356, 426) and the use of metal salts such as Cu, Fe, La, and Yb as described in U.S. Pat. 10,005,810 assigned to NuCana plc. Enzymatic-catalyzed kinetic resolution of a phosphoramidate precursor has been described in Xiang et al. (Biochemistry 2019, 58, 3204).

U.S Pat. Nos. 9,828,410; 10,000,523; 10,005,811; 10,239,911; 10,519,186; 10,815,266; 10,870,672; 10,870,673; 10,875,885; 10,894,804; and 10,906,928 and US Applications US 2021-0015841 and US 2020-0179415 assigned to Atea Pharmaceuticals disclose Compound 1 or a pharmaceutically acceptable salt of the structure below to treat hepatitis C.

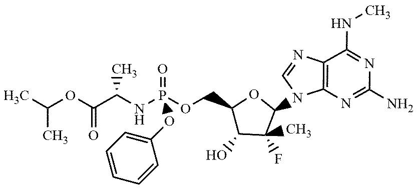 [SMILES: CNc1nc(N)nc2c1ncn2[C@@H]1O[C@H](CO[P@@](=O)(N[C@@H](C)C(=O)OC(C)C)Oc2ccccc2)[C@@H](O)[C@@]1(C)F]

In addition, U.S. Pat. No. 10,874,687 describes the use of Compound 1 to treat SARS-CoV-2, the virus that causes COVID-19.

Given the importance of Compound 1 for the therapeutic treatment of humans infected with hepatitis C or SARS-CoV-2, it would be useful to provide advantageous processes for its production.

### SUMMARY OF THE INVENTION

The present invention provides stereoselective processes for the synthesis of the purine phosphoramidate nucleotide Compound 1 wherein the Sₚ-diastereomer (i.e., the S-stereoconfiguration at the chiral phosphorus atom) is in substantially pure form, for example, in substantial excess over the Rₚ-diastereomer:

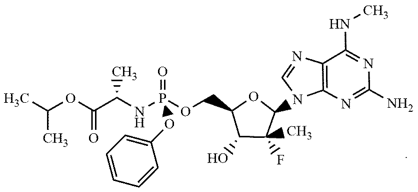

A substantially pure form of the diastereomer refers to about 90% or greater of the Sₚ- diastereomer over the Rₚ-diastereomer. In certain embodiments, the substantially pure form is about 93% pure or greater, about 95% pure or greater, about 98% pure or greater, or about 99% pure or greater, or even 100% pure. In an alternative embodiment, the substantially pure form is at least about 80%, 85%, or 90% pure.

In certain embodiments, purine Compound 1 is synthesized via a nucleophilic substitution reaction of an amino phosphorochloridate with a 3’-OH protected purine nucleoside. The protecting group at the 3’-OH position assists the direction of stereoselective addition of the phosphoramidate to the Sₚ- diastereomer.

Additional purification of the Sₚ- diastereomer can be obtained by selective crystallization in a solvent or solvent/anti-solvent system, as described in more detail below, trituration of an anti-solvent into a solvent-based solution of Compound 1, or any method known to skilled chemists that results in such purification, including column chromatography, etc. Exemplary details of the crystallization procedure are provided below. Non-limiting examples of the crystallization solvent are polar organic solvents such as an alkyl ester for example ethyl acetate, acetonitrile, DMSO, methylene chloride, acetone, or the like. Non-limiting examples of suitable anti-solvents are nonpolar organic liquids such as hydrocarbons that can be removed from the final product, including but not limited to pentane, hexane, heptane, or the like.

Importantly, in certain embodiments, this manufacturing process may be accomplished without a required extra step of protecting the N⁶-methyl, N²-amino-2,6-diaminopurine base during the reaction, which is advantageous for the efficiency of the full process. In a principal embodiment, neither amine of the N⁶-methyl or the N²-amino in the diaminopurine is protected or substantially derivatized during the process. This embodiment minimizes the need for tangential protection and/or deprotection steps.

In one aspect of the present invention, the process for synthesizing the diastereomerically pure Sₚ-phosphoramidate nucleotide of Compound 1 comprises the steps of:
(a) contacting the nucleoside compound of Formula I with isopropyl (chloro(phenoxy)phosphoryl)-Z-alaninate in the presence of an organolithium or organomagnesium reagent and optionally in the presence of an alkali halide to afford a protected diastereomerically enriched Sₚ-phosphoramidate nucleotide of Formula II wherein the Sₚ-diastereomer is in substantial excess over the Rₚ-diastereomer:

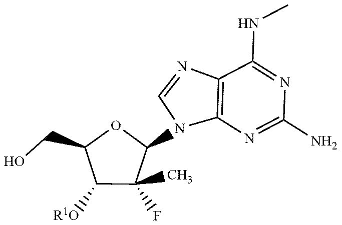

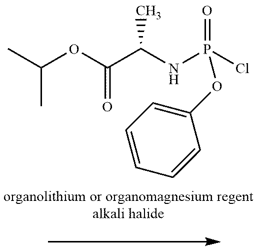

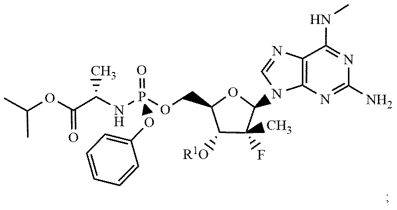

(b) further optionally purifying, for example, by selective crystallization, the diastereomerically enriched Sₚ-phosphoramidate nucleotide of Formula II to afford the diastereomerically pure Sₚ-purine phosphoramidate nucleotide of Formula II wherein the diastereomeric purity is greater than about 90%, or even greater than about 95% or even about 99% or greater; and,
(c) deprotecting the protected phosphoramidate nucleotide of Formula II to afford the diastereomerically pure Sₚ-phosphoramidate nucleotide Compound 1:

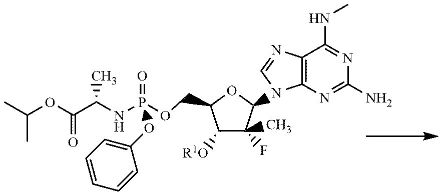

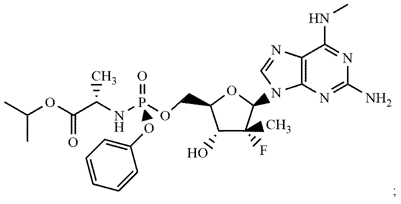

wherein R¹ is an oxygen protecting group which when attached to the oxygen is a moiety selected from a substituted benzyl ether (including para-methoxybenzyl ether (PMB)), 4-bromobenzoate, p-methoxybenzyloxymethyl ether (MPBM), o-nitrobenzyloxymethyl ether (NBOM), p-nitrobenzyloxymethyl ether, t-butoxymethyl ether, 2,2,2-trichloroethoxymethyl ether, 3-bromotetrahydropyranyl ether, tetrahydropyranyl ether, tetrahydrothiopyranyl ether, 1-methoxycyclohexyl ether, 1,4-dioxan-2-yl ether, tetrahydrofuranyl ether, tetrahydrothiofuranyl ether, a substituted phenyl ether, 2-picolyl ether, 4-picolyl ether, 1,3-benzodithiolan-2-yl ether, p-chlorophenoxyacetate ester, 3-phenylpropionate ester, p-phenylbenzoate ester, alkyl p-nitrophenyl carbonyl, alkyl benzyl carbonyl, alkylp-methoxybenzyl carbonyl, alkyl o-nitrobenzyl carbonyl, alkyl p-nitrobenzyl carbonyl, tert-butoxycarbonyl (Boc), and benzyloxycarbonyl (Cbz); and
wherein the substituent is selected from alkoxy (including but not limited to methoxy and ethoxy), hydroxy, nitro, bromo, chloro, fluoro, azido, and haloalkyl.

Nonlimiting illustrative examples of R¹ include para-methoxybenzyl ether (PMB) and tert-butoxycarbonyl (Boc).

In an alternative embodiment, the protected diastereomerically enriched Sₚ-phosphoramidate nucleotide of Formula II is first deprotected to afford the diastereomerically enriched Sₚ-phosphoramidate nucleotide Compound 1 that is then further purified, for example, via crystallization to afford the diastereomerically pure Sₚ-phosphoramidate nucleotide Compound 1. The diastereomerically enriched phosphoramidate Compound 1 prepared by this process can be a mixture of S_{p:}Rₚ diastereomers wherein the Sₚ diastereomer is in excess of the Rₚ diastereomer.

In an alternative embodiment, step (a) includes contacting the nucleoside compound of Formula I with intermediate I in the presence of an organolithium or organomagnesium reagent and optionally in the presence of an alkali halide to afford a protected diastereomerically enriched Sₚ-phosphoramidate nucleotide of Formula II wherein the Sₚ-diastereomer is in substantial excess over the Rₚ-diastereomer:

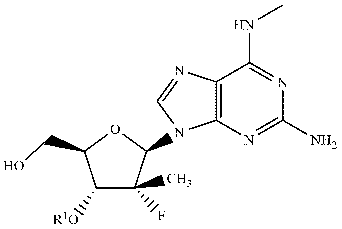

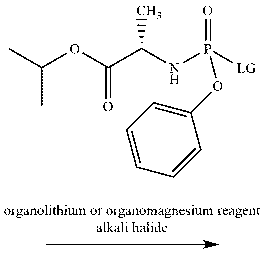

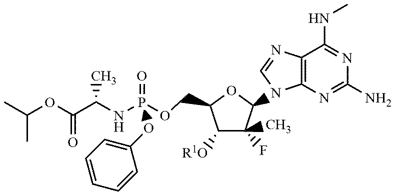

wherein
LG is a leaving group.
In certain embodiments, LG is selected from Br, I, tosylate, mesylate, trifluoroacetate, trifluorosulfonate, camphorsulfonate, triflate, acetate, —OSO₂R^{a}, aryloxide, and aryloxide substituted with at least one electron withdrawing group; and
   R^{a} is C₁₋₄alkyl, aryl, or aryl substituted with C₁₋₄alkyl, halogen, or nitro.

Non-limiting examples of electron withdrawing groups include, but are not limited to, halogen, NO₂, haloalkyl, —C(O)(alkyl), —C(O)(aryl), —C(O)O(alkyl), and —C(O)O(aryl).

Non-limiting examples of aryloxide groups substituted with at least one electron withdrawing group include p-nitrophenoxide, 2-chlorophenoxide, 4-chlorophenoxide, 2,4-dinitrophenoxide, and pentafluorophenoxide,

In certain embodiments, the ratio of S_{p:}Rₚ diastereomers in a diastereomerically enriched diastereomerically enriched phosphoramidate Compound 1 is greater than about 51:49, greater than about 55:45, greater than about 60:40, greater than about 65:35, greater than about 70:30, greater than about 75:25, greater than about 80:20, greater than about 85:15, greater than about 90:10, greater than about 95:5, greater than about 98:2, or greater than about 99:1.

In one embodiment, the purification step of the enriched mixture of Compound 1 is the selective crystallization of the enriched mixture, for example, in an alkyl acetate solvent such as ethyl acetate, a chlorinated solvent, such a dichloromethane, a ketone solvent, such as acetone, or a mixture thereof to afford pure the Sₚ phosphoramidate Compound 1. In one embodiment, the crystallization is done in an alkyl acetate solvent, for example, isopropyl acetate. In certain embodiments, the purification is conducted via selective crystallization from a solvent, for example, an alkyl acetate, a chlorinated solvent, a ketone solvent, or a mixture thereof, with an anti-solvent, for example, acetonitrile or an aliphatic hydrocarbon.

In one aspect of the present invention, the synthesis of a compound of Formula I comprises the steps of:
(1.a) the selective protection of the primary 5’ hydroxyl group on the nucleoside (3S,4R,5R)-3,4-dihydroxy-5-(hydroxymethyl)-3-methyldihydrofuran-2(3H)-one to afford a compound of Formula III wherein R² is an oxygen protecting group which when attached to the oxygen is an ester, ether, or silyl ether moiety:

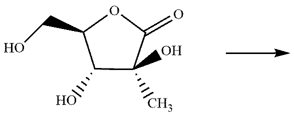

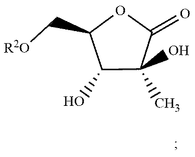

(1.b) the protection of the 3’-OH group of the nucleoside compound of Formula III with an oxygen protecting group R¹ to afford a nucleoside compound of Formula VI wherein the oxygen protecting group, R¹, assists in the direction of stereoselective addition of the phosphoramidate to the Sₚ- diastereomer:

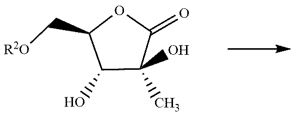

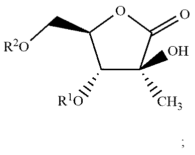

(1.c) the conversion of the alcohol of Formula IV into a monofluoride with inversion of stereochemistry to afford a compound of Formula V:

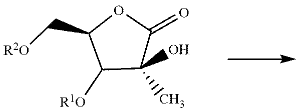

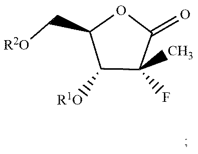

(1.d) the reduction of the lactone of the nucleoside compound of Formula V to afford the nucleoside compound of Formula VI:

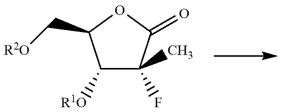

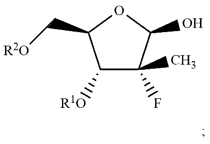

(1.e) the conversion of a compound of Formula VI to a compound of Formula VII wherein X is Cl, Br, or OAc:

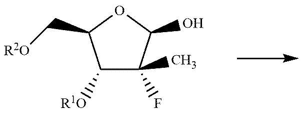

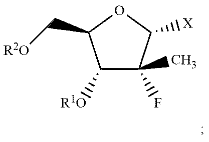

(1.f) the nucleophilic substitution of the compound of Formula VII with 2-amino-6-chloropurine to afford a compound of Formula VIII:

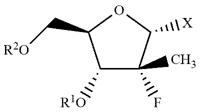

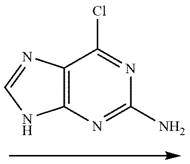 [SMILES: Nc1nc(Cl)c2nc[nH]c2n1]

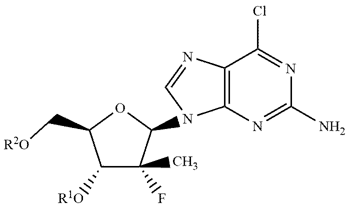

; and
(1.g) the conversion of the 2-amino-6-chloropurine base to the 2-amino-N⁶-methyl base and the selective deprotection of the 5’-position to afford a compound of Formula I:

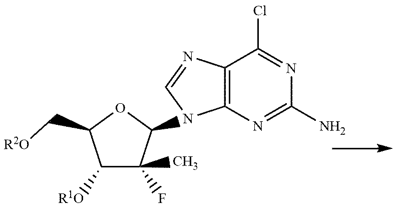

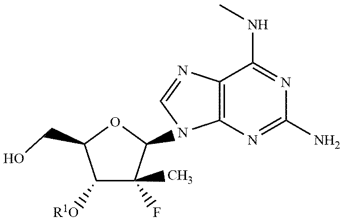

The use of bulky protecting groups at the 3’-OH position to obtain high stereoselectivity during the nucleophilic substitution step for the synthesis of pyrimidine-containing phosphoramidate nucleotides is described in Cini et al. (Eur. J. Org. Chem. 2018, 2622) and PCT Application WO 2016/151542 assigned to Quimica Sintetica, S.A.

In one embodiment, the N²-position of the nucleoside is protected prior to the phosphorylation. In this embodiment, a compound of Formula IX is reacted with isopropyl (chloro(phenoxy)phosphoryl)-Z-alaninate in the presence of an organolithium or organomagnesium reagent and optionally in the presence of an alkali halide to afford a protected diastereomerically enriched Sₚ-phosphoramidate nucleotide of Formula X wherein the Sₚ-diastereomer is in excess of the Rₚ-diastereomer:

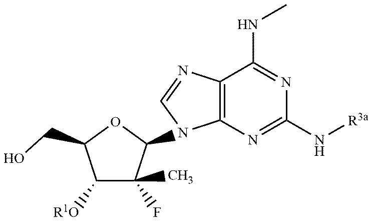

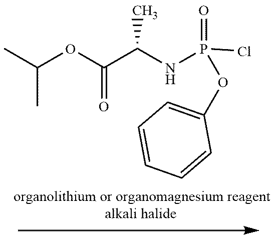

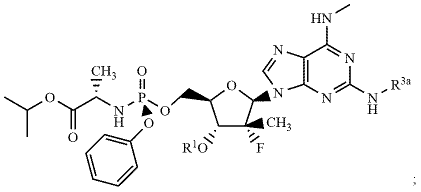

wherein R^{3a} is a nitrogen protecting group which when attached to the nitrogen is an amine, amide, or carbamate moiety and R¹ is as defined herein.

In alternative embodiments, a compound of Formula IX is reacted with Intermediate I in the presence of an organolithium or organomagnesium reagent and optionally in the presence of an alkali halide to afford a protected diastereomerically enriched Sₚ-phosphoramidate nucleotide of Formula X wherein the Sₚ-diastereomer is in excess of the Rₚ-diastereomer:

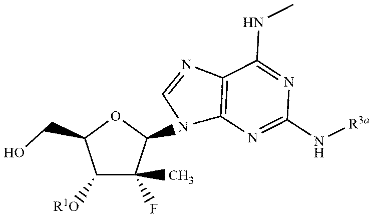

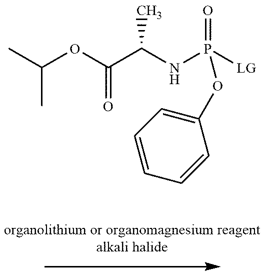

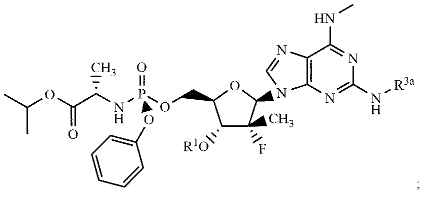

wherein R^{3a} is a nitrogen protecting group which when attached to the nitrogen is an amine, amide, or carbamate moiety and LG and R¹ are as defined herein.

In one embodiment, the synthesis of a compound of Formula IX comprises the steps:
(1.g.2) protecting the N²-position in the compound of Formula VIII with protecting group R^{3a} to afford a compound of Formula XI wherein R^{3a} is a nitrogen protecting group which when attached to the nitrogen is an amine, amide, or carbamate moiety:

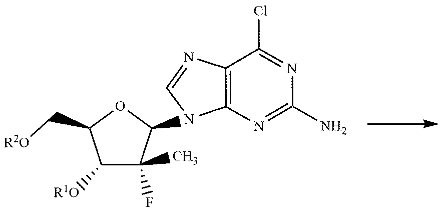

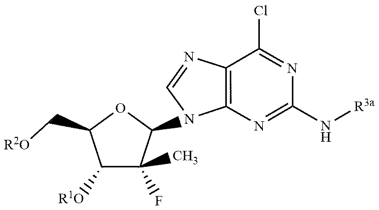

; and (1.h) converting the 6-chloro position in the compound of Formula XI to the N⁶-methylamino group and selectively deprotecting the R² position to afford a compound of Formula IX:

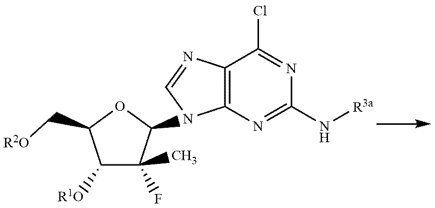

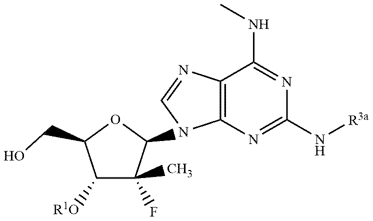

In another embodiment, the N²-amine and the N⁶-methylamine of the nucleoside are protected prior to the phosphorylation. In one embodiment, a compound of Formula XII where the N²-amine and the N⁶-methylamine are protected is reacted with isopropyl (chloro(phenoxy)phosphoryl)-Z-alaninate in the presence of an organolithium or organomagnesium reagent and optionally in the presence of an alkali halide to afford a protected diastereomerically enriched Sₚ-phosphoramidate nucleotide of Formula XIII wherein the Sₚ-diastereomer is in excess of the Rₚ-diastereomer:

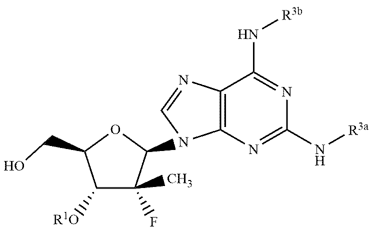

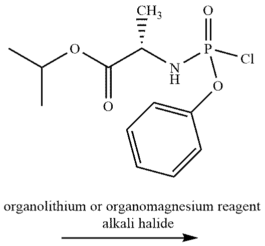

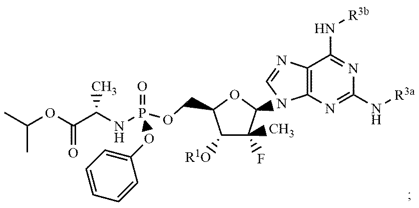

wherein R^{3b} is a nitrogen protecting group which when attached to the nitrogen is an amine, amide, or carbamate moiety and R¹ and R^{3a} are as defined herein.

In an alternative embodiment, a compound of Formula XII where the N²-amine and the N⁶-methylamine are protected is reacted with Intermediate I in the presence of an organolithium or organomagnesium reagent and optionally in the presence of an alkali halide to afford a protected diastereomerically enriched Sₚ-phosphoramidate nucleotide of Formula XIII wherein the Sₚ-diastereomer is in excess of the Rₚ-diastereomer:

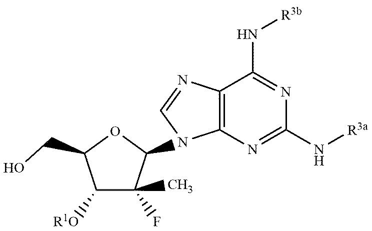

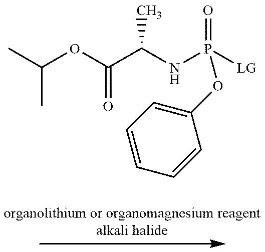

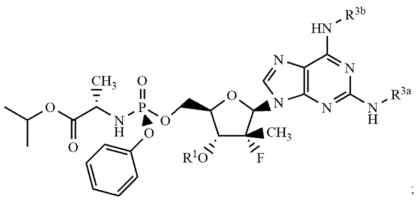

wherein R^{3b} is a nitrogen protecting group which when attached to the nitrogen is an amine, amide, or carbamate moiety and LG, R¹ and R^{3a} are as defined herein.

In one embodiment, the synthesis of a compound of Formula XII comprises protecting the N⁶-methylamine in the compound of Formula IX with protecting group R^{3b} to afford a compound of Formula XII where both the N²- and N⁶-positions are protected wherein R^{3b} is a nitrogen protecting group which when attached to the nitrogen is an amine, amide, or carbamate moiety:

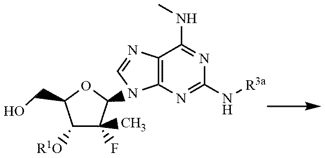

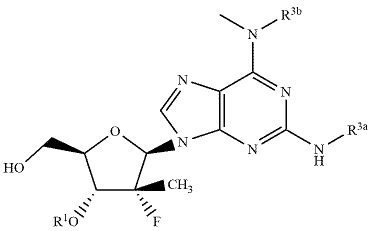

In an alternative embodiment, the N⁶-methylamine of the nucleoside is protected prior to the phosphorylation. In one embodiment, a compound of Formula XIV where the N⁶-methylamine is protected is reacted with isopropyl (chloro(phenoxy)phosphoryl)-Z-alaninate in the presence of an organolithium or organomagnesium reagent and optionally in the presence of an alkali halide to afford a protected diastereomerically enriched Sₚ-phosphoramidate nucleotide of Formula XV wherein the Sₚ-diastereomer is in excess of the Rₚ-diastereomer:

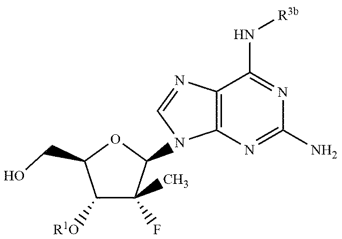

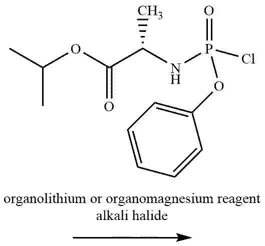

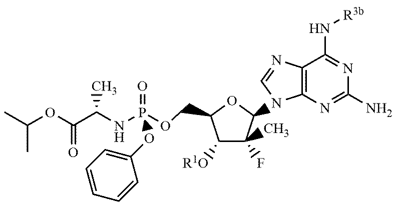

wherein R^{3b} is a nitrogen protecting group which when attached to the nitrogen is an amine, amide, or carbamate moiety and R¹ is as defined herein.

In an alternative embodiment, a compound of Formula XIV where the N⁶-methylamine is protected is reacted with Intermediate I in the presence of an organolithium or organomagnesium reagent and optionally in the presence of an alkali halide to afford a protected diastereomerically enriched Sₚ-phosphoramidate nucleotide of Formula XV wherein the Sₚ-diastereomer is in excess of the Rₚ-diastereomer:

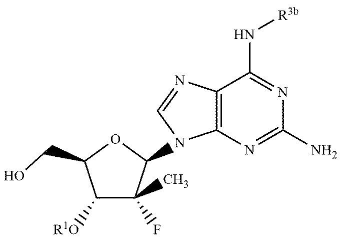

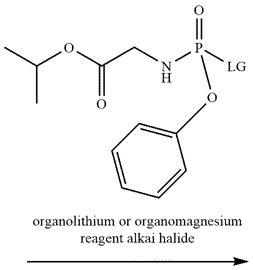

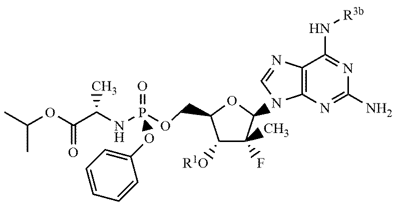

wherein R^{3b} is a nitrogen protecting group which when attached to the nitrogen is an amine, amide, or carbamate moiety and LG and R¹ are as defined herein.

In one embodiment, Formula XIV is synthesized by protecting the N⁶-methylamine in the compound of Formula I with protecting group R^{3b} to afford a compound of Formula XIV:

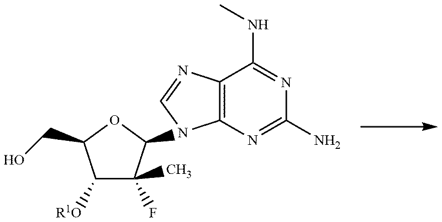

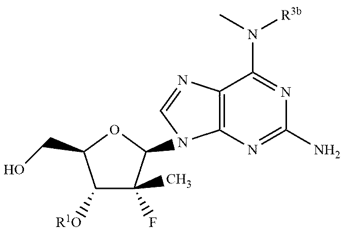

In one embodiment, R^{3a} and R^{3b} are independently nitrogen protecting groups which when attached to the nitrogen are carbamate moieties, for example, tert-butoxycarbonyl-(Boc) or benzyloxycarbonyl-(Cbz). In one embodiment, R^{3a} and R^{3b} are independently nitrogen protecting groups which when attached to the nitrogen are amine moieties, such as benzyl amine or para-methoxybenzyl amine. In one embodiment, R^{3a} and R^{3b} are similar protecting groups as R¹ and can be deprotected by a similar process as discussed herein.

In one embodiment, the protected diastereomerically enriched Sₚ-phosphoramidate nucleotides of Formula X, Formula XIII, or Formula XV are then further optionally purified, for example, by selective crystallization, to afford the diastereomerically pure Sₚ-purine phosphoramidate nucleotides of Formula X, Formula XIII, or Formula XV, respectively, wherein the diastereomeric purity is greater than about 90%, about 95% or even about 99% or greater. The diastereomerically pure Sₚ-purine phosphoramidate nucleotides of Formula X, Formula XIII, or Formula XV are then deprotected to afford the diastereomerically pure Sₚ-phosphoramidate nucleotide Compound 1.

In one embodiment, Compound 1 and Compound 1-A are prepared via the synthesis below wherein R¹, when attached to the oxygen, is a substituted benzyl ether protecting group, for example, para-methoxybenzyl ether:

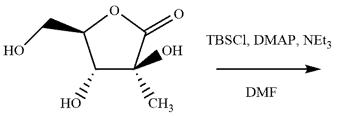

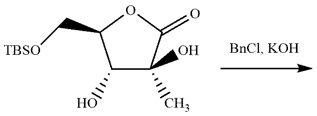

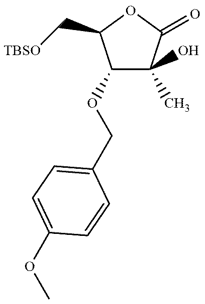 [SMILES: COc1ccc(CO[C@@H]2[C@@H](CO[Si](C)(C)C(C)(C)C)OC(=O)[C@@]2(C)O)cc1]

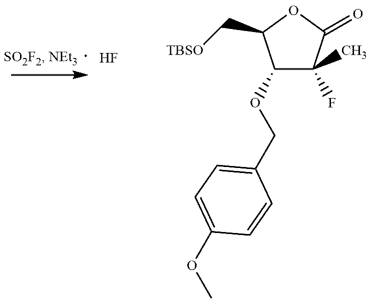

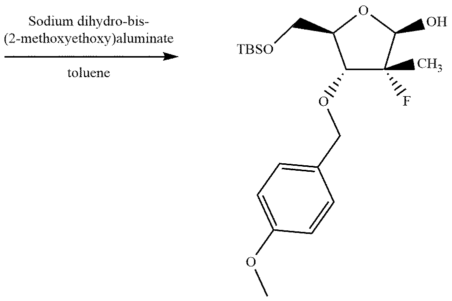

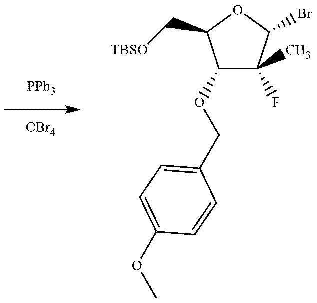 [SMILES: "CBr4" ; COc1ccc(CO[C@@H]2[C@@H](CO[Si](C)(C)C(C)(C)C)O[C@H](Br)[C@]2(C)F)cc1 ; "PPh3"]

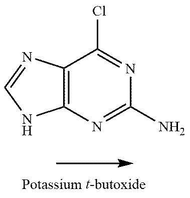

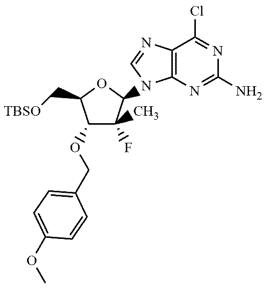 [SMILES: COc1ccc(CO[C@@H]2[C@@H](CO[Si](C)(C)C(C)(C)C)O[C@@H](n3cnc4c(Cl)nc(N)nc43)[C@]2(C)F)cc1]

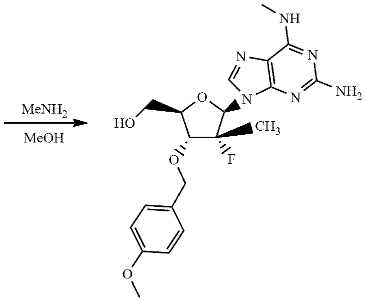 [SMILES: "MeNH2" ; "MeOH" ; CNc1nc(N)nc2c1ncn2[C@@H]1O[C@H](CO)[C@@H](OCc2ccc(OC)cc2)[C@@]1(C)F]

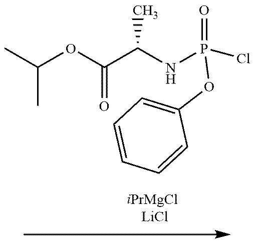

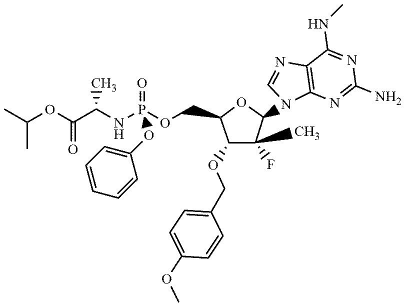 [SMILES: CNc1nc(N)nc2c1ncn2[C@@H]1O[C@H](CO[P@@](=O)(N[C@@H](C)C(=O)OC(C)C)Oc2ccccc2)[C@@H](OCc2ccc(OC)cc2)[C@@]1(C)F]

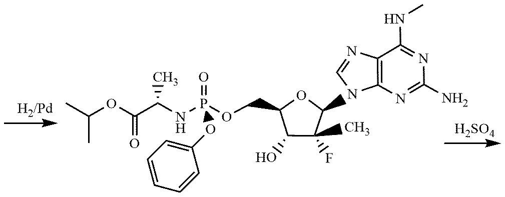

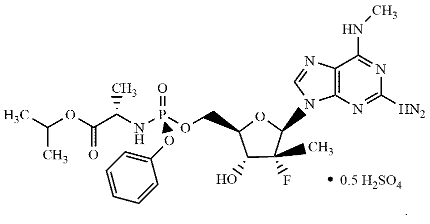

In another aspect, the present invention provides nucleoside compounds of Formula I, Formula IX, Formula XII, and Formula XIV:

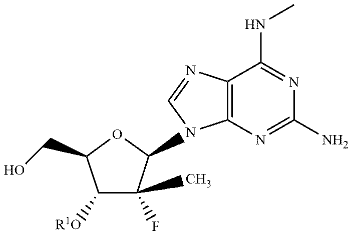

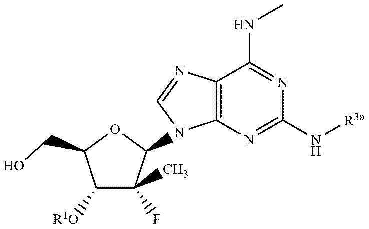

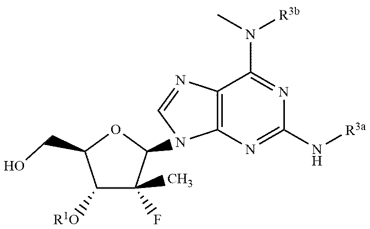

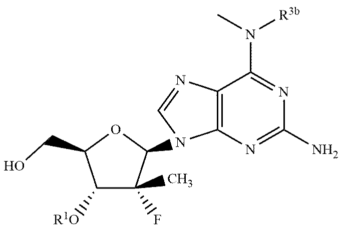

wherein R¹, R^{3a}, and R^{3b} are as defined herein.

### DETAILED DESCRIPTION OF THE INVENTION

The present invention provides stereoselective processes for the synthesis of the purine phosphoramidate nucleotide Compound 1 wherein the Sₚ-diastereomer is in a substantially pure form, for example, in excess over the Rₚ-diastereomer:

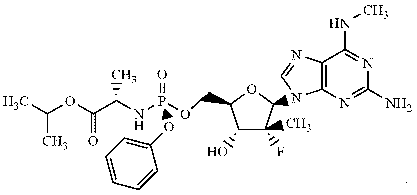

A substantially pure form of the diastereomer refers to about 90% or greater of the Sₚ- diastereomer over the Rₚ-diastereomer. In certain embodiments, the substantially pure form is about 93% pure or greater, about 95% pure or greater, about 98% pure or greater, about 99% pure or greater, or even 100% pure. In an alternative embodiment, a substantially pure form of the diastereomer refers to about 80%, 85%, or 90% of the Sₚ- diastereomer over the Rₚ-diastereomer.

In one embodiment, purine Compound 1 is synthesized via a nucleophilic substitution reaction of an amino phosphorochloridate or Intermediate I with a 3’-OH protected purine nucleoside. The protecting group at the 3’-OH position assists the direction of stereoselective addition of the phosphoramidate toward the Sₚ- diastereomer.

In one embodiment, Compound 1 is prepared as a pharmaceutically acceptable salt, for example, by reaction with a pharmaceutically acceptable acid, as described more fully herein.

In one embodiment, the pharmaceutically acceptable salt form of Compound 1 is the hemi-sulfate salt form, Compound 1-A:

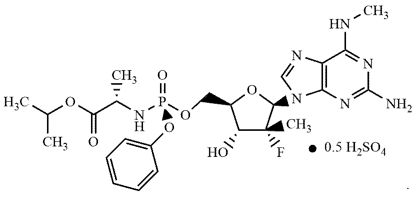

In one embodiment, Compound 1-A is prepared from Compound 1 by the dropwise addition of concentrated H₂SO₄ in MeOH and the filtration of the resulting precipitate.

In one aspect of the present invention, the process for synthesizing the diastereomerically pure Sₚ-phosphoramidate nucleotide of Compound 1 comprises the steps of:
(a) contacting the nucleoside compound of Formula I with isopropyl (chloro(phenoxy)phosphoryl)-Z-alaninate in the presence of an organolithium or organomagnesium reagent and optionally in the presence of an alkali halide to afford a protected diastereomerically enriched Sₚ-phosphoramidate nucleotide of Formula II wherein the Sₚ-diastereomer is in excess of the Rₚ-diastereomer:

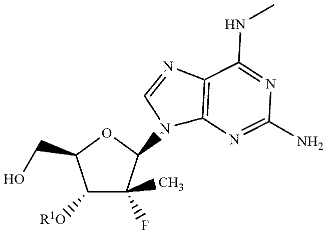

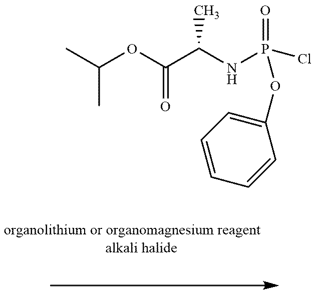

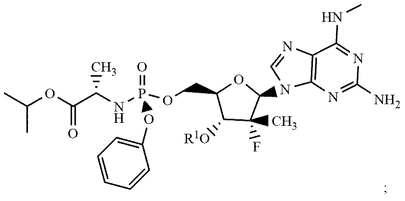

(b) further optionally purifying, as a nonlimiting example, by selective crystallization, the diastereomerically enriched Sₚ-phosphoramidate nucleotide of Formula II to afford the diastereomerically pure Sₚ-purine phosphoramidate nucleotide of Formula II wherein the diastereomeric purity is greater than about 90%, greater than about 95% or even about 99% or greater; and,
(c) deprotecting the protected phosphoramidate nucleotide of Formula II to afford the diastereomerically pure Sₚ-phosphoramidate nucleotide Compound 1:

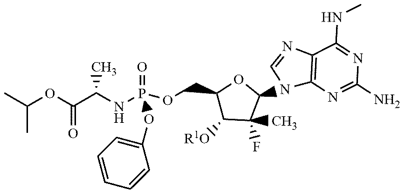

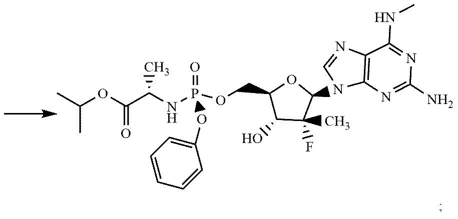

wherein R¹ is an oxygen protecting group which when attached to the oxygen is a moiety selected from a substituted benzyl ether (including para-methoxybenzyl ether), 4-bromobenzoate, p-methoxybenzyloxymethyl ether (MPBM), o-nitrobenzyloxymethyl ether (NBOM), p-nitrobenzyloxymethyl ether, t-butoxymethyl ether, 2,2,2-trichloroethoxymethyl ether, 3-bromotetrahydropyranyl ether, tetrahydropyranyl ether, tetrahydrothiopyranyl ether, 1-methoxycyclohexyl ether, 1,4-dioxan-2-yl ether, tetrahydrofuranyl ether, tetrahydrothiofuranyl ether, a substituted phenyl, 2-picolyl ether, 4-picolyl ether, 1,3-benzodithiolan-2-yl ether, p-chlorophenoxyacetate ester, 3-phenylpropionate ester, p-phenylbenzoate ester, alkyl p-nitrophenyl carbonyl, alkyl benzyl carbonyl, alkylp-methoxybenzyl carbonyl, alkyl o-nitrobenzyl carbonyl, alkyl p-nitrobenzyl carbonyl, t-butylcarbonyl (Boc), and benzylcarbonyl (Cbz); and
wherein the substituent is selected from alkoxy (including methoxy and ethoxy), hydroxy, nitro, bromo, chloro, fluoro, azido, and haloalkyl.

In an alternative embodiment, step (a) includes contacting the nucleoside compound of Formula I with Intermediate I in the presence of an organolithium or organomagnesium reagent and optionally in the presence of an alkali halide to afford a protected diastereomerically enriched Sₚ-phosphoramidate nucleotide of Formula II wherein the Sₚ-diastereomer is in substantial excess over the Rₚ-diastereomer:

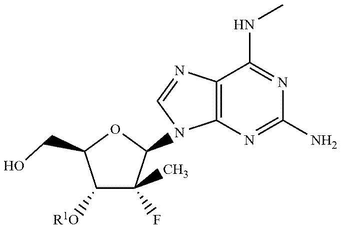

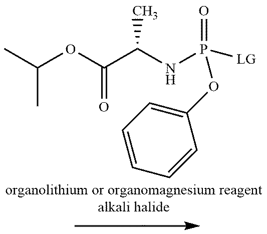

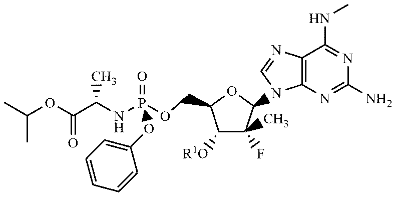

wherein LG is a leaving group.

In certain embodiments, LG is selected from Br, I, tosylate, mesylate, trifluoroacetate, trifluorosulfonate, camphorsulfonate, triflate, acetate, —OSO₂R^{a}, aryloxide, and aryloxide substituted with at least one electron withdrawing group; and

R^{a} is C₁₋₄alkyl, aryl, or aryl substituted with C₁₋₄alkyl, halogen, or nitro.

Non-limiting examples of electron withdrawing groups include, but are not limited to, halogen, NO₂, haloalkyl, —C(O)(alkyl), —C(O)(aryl), —C(O)O(alkyl), and —C(O)O(aryl).

Non-limiting examples of aryloxide groups substituted with at least one electron withdrawing group include p-nitrophenoxide, 2-chlorophenoxide, 4-chlorophenoxide, 2,4-dinitrophenoxide, and pentafluorophenoxide.

Additional optional steps include:
(d) further optionally purifying Compound 1; and
(e) preparing the pharmaceutically acceptable salt form of the diastereomerically pure Sₚ-purine phosphoramidate nucleotide Compound 1.

Non-limiting examples of organolithium reagents include methyllithium, n-butyllithium, sec-butyllithium, isopropyllithium, and tert-butyllithium. Organomagnesium reagents have the general formula R'-Mg-X' wherein R′ is a halogen and X′ is alkyl or aryl. Non-limiting examples of organomagnesium reagents include tert-butylmagnesium chloride (tBuMgCl), iso-propylmagnesium chloride (iPrMgCl), ethylmagnesium chloride, methyl magnesium chloride, phenylmagnesium chloride, tert-butylmagnesium bromide (tBuMgBr), iso-propylmagnesium bromide (iPrMgBr), ethylmagnesium bromide, methylmagnesium bromide, and phenylmagnesium bromide.

The nucleophilic substitution is optionally conducted in the presence of an alkali halide, for example lithium chloride, sodium chloride, potassium chloride, or cesium chloride.

In an alternative embodiment, the protected diastereomerically enriched Sₚ-phosphoramidate nucleotide of Formula II is first deprotected to afford the diastereomerically enriched Sₚ-phosphoramidate nucleotide Compound 1 that is then further purified, for example, via crystallization to afford the diastereomerically pure Sₚ-phosphoramidate nucleotide Compound 1. The diastereomerically enriched phosphoramidate Compound 1 prepared by this process can be a mixture of S_{p:}Rₚ diastereomers wherein the Sₚ diastereomer is in excess of the Rₚ diastereomer.

In an alternative embodiment, step (b) affords diastereomerically pure Sₚ-purine phosphoramidate nucleotide of Formula II wherein the diastereomeric purity is greater than about 80% or is 85%-90% pure.

Step (a) affords a diastereomerically enriched phosphoramidate of Formula II wherein the Sₚ-diastereomer is in excess of the Rₚ-diastereomer. In certain embodiments, the ratio of S_{p:}Rₚ diastereomers in a diastereomerically enriched phosphoramidate compound of Formula II is greater than about 51:49, greater than about 55:45, greater than about 60:40, greater than about 65:35, greater than about 70:30, greater than about 75:25, greater than about 80:20, greater than about 85:15, greater than about 90:10, greater than about 95:5, greater than about 98:2, or greater than about 99:1.

In certain embodiments, the purification in step (b) is the selective crystallization of the enriched mixture, for example, in an alkyl acetate solvent such as ethyl acetate, a chlorinated solvent, such a dichloromethane, a ketone solvent, such as acetone, or a mixture thereof to afford pure a Sₚ phosphoramidate compound of Formula II. In one embodiment, the crystallization is conducted in an alkyl acetate, such as isopropyl acetate. In certain embodiments, the purification is conducted via selective crystallization from an alkyl acetate, chlorinated solvent, or a mixture thereof, with acetonitrile or an aliphatic hydrocarbon.

In one embodiment, the purification in step (b) is the selective crystallization of the enriched mixture wherein the enriched mixture is dissolved in an organic solvent and then an anti-solvent is added dropwise to the above solution system wherein the organic solvent comprises, for example, a solvent selected from C₁-₈ alcohol(s), C₂-₈ ether(s), C₃-₇ ketone(s), C₃-₇ ester(s), C₁-₂ chlorocarbon(s), and C₂-₇ nitrile(s) or a mixture thereof and wherein the anti-solvent comprises a solvent that is not substantially miscible with the solvent, such as a C₅-₁₂ saturated hydrocarbon(s), C₆-₁₂ aromatic hydrocarbon(s), or petroleum ether. In certain embodiments, the organic solvent is selected from ethyl acetate, tert-butyl methyl ether, isopropanol and tetrahydrofuran. In an alternative embodiment, the organic solvent is isopropyl acetate. In some embodiments, the anti-solvent is selected from petroleum ether or hexane.

The diastereomerically pure compound of Formula II, for example may be greater than about 95% pure, greater than about 96%, greater than about 98%, greater than about 99%, or 100% pure. In an alternative embodiment, the diastereomerically pure compound of Formula II is greater than 80% or is 85-90% pure.

The deprotection conditions of Formula II are those generally known to the skilled artisan and are described, for example, in Theodora W. Green, Protective Groups in Organic Synthesis, Third Edition, John Wiley & Sons (1999) or Theodora W. Green, Protective Groups in Organic Synthesis, Fifth Edition, John Wiley & Sons (2014), which are discussed below. When R¹ is a protecting group which when attached to the oxygen is a substituted benzyl ether, Formula II can, for example, be subjected to conditions described on pages 86-101 of the Third Edition. When R¹ is a protecting group, for example, which when attached to the oxygen is a para-methoxybenzyl ether, two non-limiting examples of deprotection conditions include, for example, DDQ (2,3-dichloro-5,6-dicyano-1,4-benzoquinone) in CH₂Cl₂ and hydrogenolysis (H2/Pd-C).

In alternative embodiments, the protected diastereomerically enriched Sₚ-phosphoramidate nucleotide of Formula II is first deprotected to afford the diastereomerically enriched Sₚ-phosphoramidate nucleotide Compound 1 that is then further purified, for example, via crystallization to afford the diastereomerically pure Sₚ-phosphoramidate nucleotide Compound 1. The diastereomerically enriched phosphoramidate Compound 1 prepared by this process can be a mixture of S_{p:}Rₚ diastereomers wherein the Sₚ diastereomer is in excess of the Rₚ diastereomer. In certain embodiments, the ratio of S_{p:}Rₚ diastereomers in a diastereomerically enriched phosphoramidate Compound 1 is greater than about 51:49, greater than about 55:45, greater than about 60:40, greater than about 65:35, greater than about 70:30, greater than about 75:25, greater than about 80:20, greater than about 85:15, greater than about 90:10, greater than about 95:5, greater than about 98:2, or greater than about 99:1.

In this aspect, the purification step of the enriched mixture of Compound 1 is crystallization of the enriched mixture, for example, in an alkyl acetate solvent such as ethyl acetate, a chlorinated solvent, such a dichloromethane, a ketone solvent, such as acetone, or a mixture thereof to afford pure the Sₚ phosphoramidate Compound 1. In certain aspects, the purification is conducted via crystallization from an alkyl acetate, chlorinated solvent, or a mixture thereof, with acetonitrile or an aliphatic hydrocarbon.

In aspects of the present invention, the synthesis of a compound of Formula I comprises the steps of:
(1.a) the selective protection of the primary 5’-hydroxyl group on the nucleoside (3S,4R,5R)-3,4-dihydroxy-5-(hydroxymethyl)-3-methyldihydrofuran-2(3H)-one to afford a compound of Formula III wherein R² is an oxygen protecting group which when attached to the oxygen is an ester, ether, or silyl ether moiety:

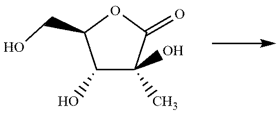

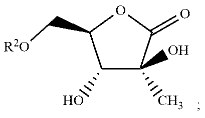

(1.b) the protection of the 3’-OH group of the nucleoside compound of Formula III with an oxygen protecting group R¹ to afford a nucleoside compound of Formula VI wherein the oxygen protecting group, R¹, assists in the direction of stereoselective addition of the phosphoramidate toward the Sₚ- diastereomer:

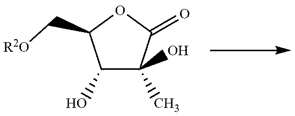

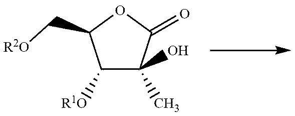

(1.c) the conversion of the alcohol of Formula IV into a monofluoride with inversion of stereochemistry to afford a compound of Formula V:

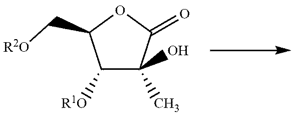

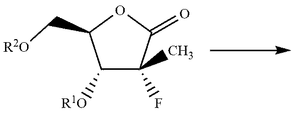

(1.d) the reduction of the lactone of the nucleoside compound of Formula V to afford the nucleoside compound of Formula VI:

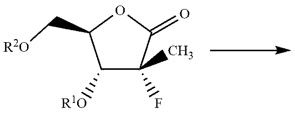

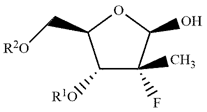

(1.e) the conversion of a compound of Formula VI to a compound of Formula VII wherein X is Cl, Br, or OAc:

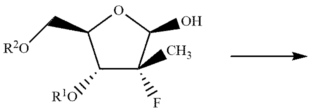

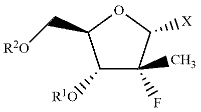

(1.f) the nucleophilic substitution of the compound of Formula VII with 2-amino-6-chloropurine to afford a compound of Formula VIII:

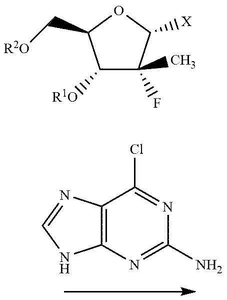

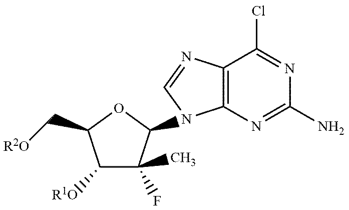

; and (1.g) the conversion of the 2-amino-6-chloropurine base to the 2-amino-N⁶-methyl base and the selective deprotection of the 5’-position to afford a compound of Formula I:

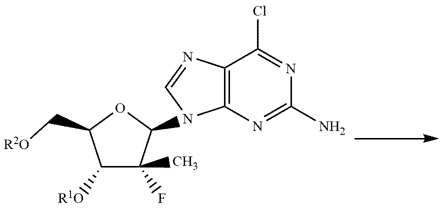

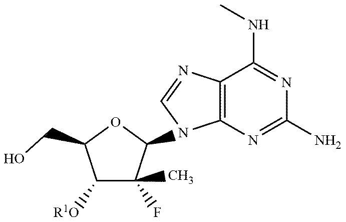

Step (1.a) comprises the selective protection of the primary 5’-hydroxyl group on the nucleoside (3S,4R,5R)-3,4-dihydroxy-5-(hydroxymethyl)-3-methyldihydrofuran-2(3H)-one with an oxygen protecting group R² that when attached to the oxygen is an ether, ester, or silyl ether moiety.

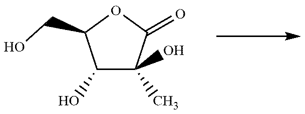

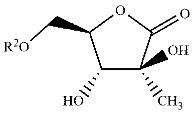

This step may be conducted according to one of the procedures described in Theodora W. Green, Protective Groups in Organic Synthesis, Third Edition, John Wiley & Sons (1999) or Theodora W. Green, Protective Groups in Organic Synthesis, Fifth Edition, John Wile0y & Sons (2014) for the protection of hydroxyls.

In one embodiment, R² is an oxygen protecting group that when attached to the oxygen is an ester moiety, for example benzoate or acetate. When R² is an oxygen protecting group which when attached to the oxygen is an ester moiety, the compound of Formula III can be prepared according to the conditions described in the Fifth Edition of Protective Groups in Organic Synthesis on pages 271-374. For example, an acetate group can be installed using Ac₂O in pyridine or Ac₂O and imidazole in CH₃CN as described on page 273. A benzoate ester can be installed using BzCl or Bz₂O in pyridine at 0° C. or BzCl and LiClO₄ in THF as described on page 316.

In one embodiment, R² is an oxygen protecting group that when attached to the oxygen is a silyl ether moiety (for example (trimethylsilyl (TMS), triisopropylsilyl (TIPS), tertbutyldimethylsilyl (TBDMS or TBS) or tert-butyldiphenylsilyl (TBDPS). When R² is a silyl ether moiety when attached to the oxygen, the compound of Formula III can be prepared according to the conditions described in Theodora W. Green, Protective Groups in Organic Synthesis, Third Edition, John Wiley & Sons (1999) on pages 113-147. In one embodiment, R² is a tert-butyldimethylsilyl (TBS) group. The TBS group is selectively installed on the primary alcohol over the secondary alcohol using the conditions described in the text on page 128 and in Ogilvie et al. Can. J. Chem. 1979, 57, 2230. These conditions include the use of TBSC1, DMAP, and NEt₃ in DMF at 25° C.

In certain embodiments, R² is an oxygen protecting group that when attached to the oxygen is an ether moiety, for example methyl ether, methoxymethyl ether, or benzyl ether.
Step (1.b) includes the protection of the hydroxyl group in the 3’-postion with a protecting group R¹ the assists in the direction of stereoselective addition of the phosphoramidate toward the Sₚ- diastereomer:

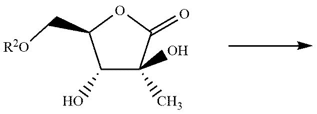

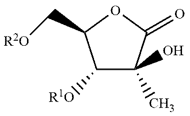

In certain aspects, R¹ is an oxygen protecting group which when attached to the oxygen is a moiety selected from a substituted benzyl ether wherein the substituent is selected from alkoxy, hydroxy, nitro, bromo, chloro, fluoro, azido, and haloalkyl.

Non-limiting examples of substituted benzyl ether moieties include p-methoxybenzyl, 3,4-dimethoxybenzyl, 2,4-dimethoxybenzyl, 2-hydroxybenzy, 3,4-dimethoxybenzyl, 2,3,4-trimethoxybenzyl, 3,4,5-trimethoxybenzyl, 2,5-dimethoxybenzyl, o-nitrobenzyl, p-nitrobenzyl, p-bromobenzyl, p-chlorobenzyl, 2,6-dichlorobenzyl, p-phenylbenzyl, 2,6-difluorobenzyl, p-azidobenzyl, 2-trifluorobenzyl, and 4-azido-3-chlorobenzyl.

In certain embodiments, R¹ is an oxygen protecting group which when attached to the oxygen is an ether moiety, which for example is selected from p-methoxybenzyloxymethyl ether (MPBM), o-nitrobenzyloxymethyl ether (NBOM), p-nitrobenzyloxymethyl ether, t-butoxymethyl ether, 2,2,2-trichloroethoxymethyl ether, 3-bromotetrahydropyranyl ether, tetrahydropyranyl ether, tetrahydrothiopyranyl ether, 1-methoxycyclohexyl ether, 1,4-dioxan-2-yl ether, tetrahydrofuranyl ether, tetrahydrothiofuranyl ether, a substituted phenyl ether, 2-picolyl ether, 4-picolyl ether, and 1,3-benzodithiolan-2-yl ether.

In certain embodiments, R¹ is an oxygen protecting group which when attached to the oxygen is an ester moiety selected from p-chlorophenoxyacetate ester, 3-phenylpropionate ester, and p-phenylbenzoate ester.

In certain embodiments, R¹ is an oxygen protecting group which when attached to the oxygen is a carbonate protecting group selected from alkyl p-nitrophenyl carbonyl, alkyl benzyl carbonyl, alkyl p-methoxybenzyl carbonyl, alkyl o-nitrobenzyl carbonyl, alkyl p-nitrobenzyl carbonyl, t-butylcarbonyl (Boc), and benzylcarbonyl (Cbz).

This step may also be conducted according to one of the procedures described in Theodora W. Green, Protective Groups in Organic Synthesis, Fifth Edition, John Wiley & Sons (2014). For example, when R¹ is an oxygen protecting group which when attached to the oxygen is a substituted benzyl ether, the compound of Formula II can be prepared according to the conditions described in the text on pages 120-126, including, for example, BnCl in the presence of KOH or BnBr in (i-Pr)₂EtN.

Step (1.c), in a principle embodiment, includes the conversion of the hydroxyl group in the compound of Formula IV to the corresponding monofluoride primarily with inversion of stereochemistry to afford a compound of Formula V (“α-fluoro” configuration):

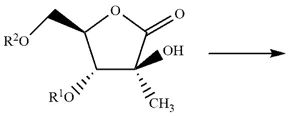

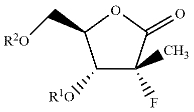

In embodiments, step (1.c) is conducted with a sulfonyl fluoride/TREAT•HF mixture (SO₂F₂, NEt₃•HF). In embodiments, step (1.c) is conducted with DAST (Et₂NSF₃). In one embodiment, step (1.c) is conducted with Deoxo-Fluor®. In alternative embodiments, step (1.c) as described herein is conducted with morpholinosulfur trifluoride (Morph-DAST).

In alternative embodiments, the fluorination reaction primarily proceeds with retention of stereochemistry at the 2’-position. In this embodiment, a compound of Formula IV’ is reacted with a fluorination reagent to afford a compound of Formula V:

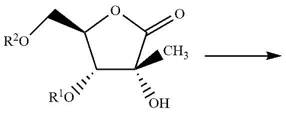

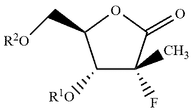

If the product of the fluorination reaction is a mixture of “α-fluoro” and “β-fluoro” lactone derivatives, the compounds can be separated by conventional methods known to a skilled artisan, for example, column chromatography or crystallization, to isolate the desired stereochemistry (“α-fluoro” configuration).

Additional non-limiting examples of nucleophilic fluorination reagents include pyridinium poly(hydrogen fluoride) (Olah’s reagent), nitrosonium tetrafluoroborate/pyridinium poly(hydrogen fluoride), triethylamine tris(hydrogen fluorine) (TREAT•HF), perfluoro-1-butanesulfonyl fluoride (PBSF), Yarovenko’s reagent, Ishikawa’s reagent, TFEDMA, N,N′-dimethyl-2,2,-difluroimidazolidine, 4-morpholinosulfur trifluoride, bromine trifluoride, and 4-tert-butyl-2,6-dimethylphenylsulfur trifluoride (Fluolead™). The fluorination reaction can be conducted according to conditions described in Pankiewicz, K., Journal of Fluorine Chemistry, 1993, 64, 15-36; Hudlicky, M. “Fluorination with Diethylaminosulfur Trifluoride and related Aminofluorosulfuranes” in Organic Reactions, Vol. 35, 1998, 513-637; Singh et al. Synthesis, 2002, 17, 2561-2578; and, Liang, Theresa, et al. Angewandte Chemie International Edition, 2013, 52, 8214-8264.

In additional embodiments, the N²-position of the nucleoside is protected prior to the phosphorylation. In these embodiments, a compound of Formula IX where the N²-amine is protected is reacted with, for example, isopropyl (chloro(phenoxy)phosphoryl)-L-alaninate in the presence of an organolithium or organomagnesium reagent and optionally in the presence of an alkali halide to afford a protected diastereomerically enriched Sₚ-phosphoramidate nucleotide of Formula X wherein the Sₚ-diastereomer is in excess of the Rₚ-diastereomer:

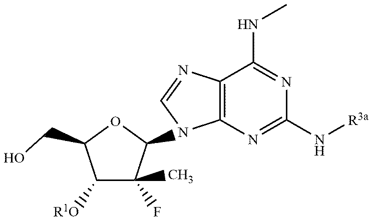

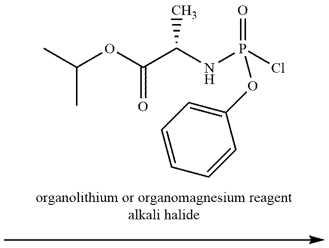

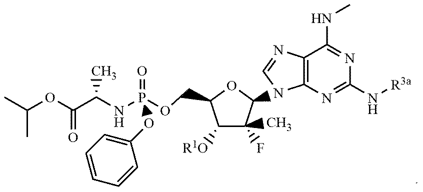

In one embodiment, the synthesis of a compound of Formula IX comprises the steps:
(1.g.2) protecting the N²-position in the compound of Formula VIII with protecting group R^{3a}to afford a compound of Formula XI wherein R^{3a} is a nitrogen protecting which when attached to the nitrogen is an amine, amide, or carbamate moiety:

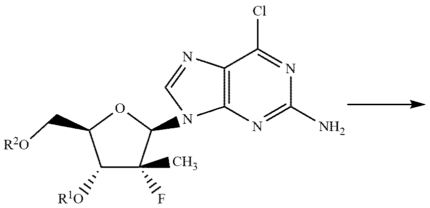

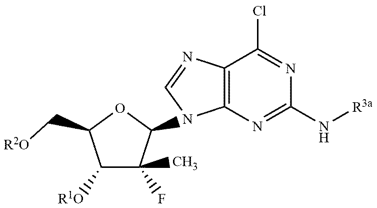

; and
(1.h) converting the 6-chloro position in the compound of Formula XI to the N⁶-methylamino group and selectively deprotecting the R¹ position to afford a compound of Formula IX:

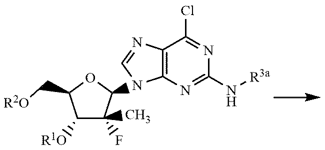

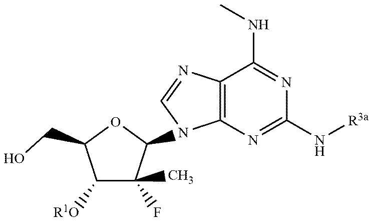

In an additional alternative embodiment, the N²-amine and the N⁶-methylamine of the nucleoside are protected prior to the phosphorylation. In this embodiment, a compound of Formula XII where the N²-amine and the N⁶-methylamine are protected is reacted with isopropyl (chloro(phenoxy)phosphoryl)-Z-alaninate in the presence of an organolithium or organomagnesium reagent and optionally in the presence of an alkali halide to afford a protected diastereomerically enriched Sₚ-phosphoramidate nucleotide of Formula XIII wherein the Sₚ-diastereomer is in excess of the Rₚ-diastereomer:

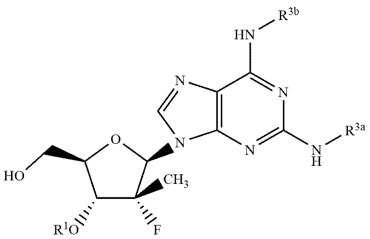

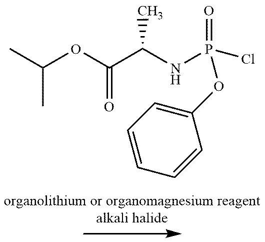

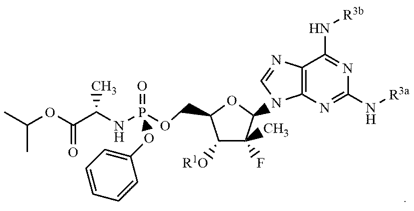

In some embodiments, the synthesis of a compound of Formula XII includes protecting the N⁶-methylamine in the compound of Formula IX with protecting group R^{3b}to afford a compound of Formula XII wherein R^{3b} is a nitrogen protecting group which when attached to the nitrogen is an amine, amide, or carbamate moiety:

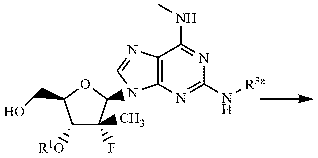

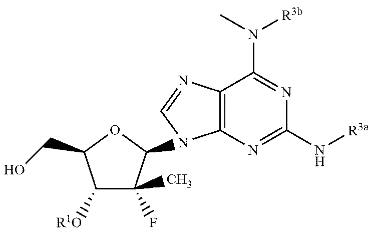

In an alternative embodiment, the N⁶-methylamine of the nucleoside is protected prior to phosphorylation. In this embodiment, a compound of Formula XIV where the N⁶-methylamine is protected is reacted, for example, with isopropyl (chloro(phenoxy)phosphoryl)-Z-alaninate in the presence of an organolithium or organomagnesium reagent and optionally in the presence of an alkali halide to afford a protected diastereomerically enriched Sₚ-phosphoramidate nucleotide of Formula XV wherein the Sₚ-diastereomer is in excess of the Rₚ-diastereomer:

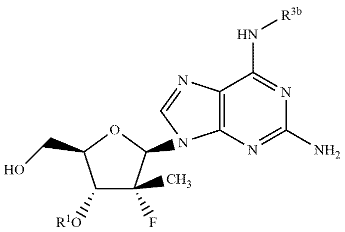

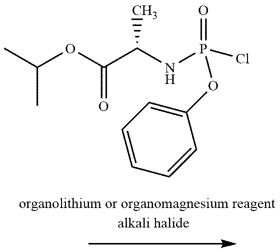

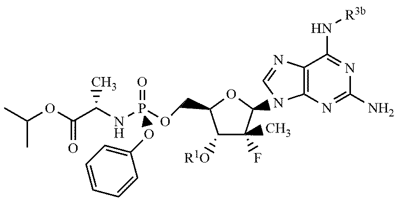

In some aspects, Formula XIV is synthesized by protecting the N⁶-methylamine in the compound of Formula I with protecting group R^{3b} to afford a compound of Formula XIV where the N⁶-methylamine positions:

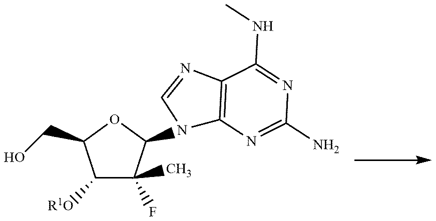

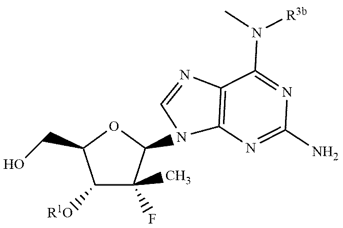

In alternative embodiments, a compound of Formula IX, XII, and XIV are reacted with Intermediate I in the presence of an organolithium or organomagnesium reagent and optionally in the presence of an alkali halide to afford a protected diastereomerically enriched Sₚ-phosphoramidate nucleotide of Formula II wherein the Sₚ-diastereomer is in substantial excess over the Rₚ-diastereomer.

In certain embodiments, R^{3a} and R^{3b} are independently nitrogen protecting groups which when attached to the nitrogen are carbamate moieties, for example, tert-butoxycarbonyl-(Boc) or benzyloxycarbonyl-(Cbz).

In certain embodiments, R^{3a} and R^{3b} are independently nitrogen protecting groups which when attached to the nitrogen are amine moieties, for example, benzyl amine or para-methoxybenzyl amine.

In certain embodiments, R^{3a} and R^{3b} are similar protecting groups to R¹ and can be deprotected by a similar process as discussed herein.

In certain embodiments, R^{3a} and R^{3b} are independently benzyl amines when attached to the nitrogen. The benzyl group can be formed and cleaved as described in Theodora W. Green, Protective Groups in Organic Synthesis, Third Edition, John Wiley & Sons (1999) on pages 579-580. For example, the benzyl group can be installed using BnBr and NEt₃ in CH₃CN and the benzyl group can be removed with Pd/C and HCOOH in CH₃OH.

In certain embodiments, R^{3a} and R^{3b} are independently a tert-butoxycarbonyl-(Boc) group that is formed and cleaved as described in Theodora W. Green, Protective Groups in Organic Synthesis, Third Edition, John Wiley & Sons (1999) on pages 518-525.

The protected diastereomerically enriched Sₚ-phosphoramidate nucleotides of Formula X, Formula XIII, or Formula XV can then be then further optionally purified, for example, by selective crystallization, to afford the diastereomerically pure Sₚ-purine phosphoramidate nucleotides of Formula X, Formula XIII, or Formula XV, respectively, wherein the diastereomeric purity is greater than about 90%, about 95% or even about 99% or greater; and then deprotected to afford the diastereomerically pure Sₚ-phosphoramidate nucleotide Compound 1. In an alternative embodiment, the diastereomerically pure Sₚ-purine phosphoramidate nucleotides of Formula X, Formula XIII, or Formula XV, have a diastereomeric purity greater than about 80% or is 85-90% pure.

Compound 1 can optionally be further purified and/or converted to a pharmaceutically acceptable salt, for example Compound 1-A.

A process is also provided for the synthesis of a Sₚ-phosphoramidate nucleoside other than the specific phosphoramidate described in the compound illustration. In one embodiment, a process is provided for the synthesis of a phosphoramidate of Formula XVI wherein the Sₚ-isomer is in excess of the Rₚ-isomer:

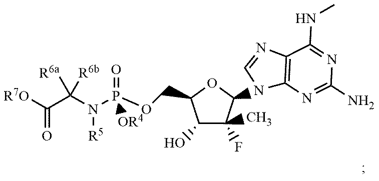

or a pharmaceutically acceptable salt thereof;
wherein:
R⁴ is hydrogen, C₁-₆alkyl (including methyl, ethyl, propyl, and isopropyl), C₃-₇cycloalkyl, or aryl (including phenyl and napthyl);
R⁵ is hydrogen or C₁-₆alkyl (including methyl, ethyl, propyl, and isopropyl);
R^{6a} and R^{6b} are independently selected from hydrogen, C₁-₆alkyl (including methyl, ethyl, propyl, and isopropyl), or C₃-₇cycloalkyl; and
R⁷ is hydrogen, C₁-₆alkyl (including methyl, ethyl, propyl, and isopropyl), C₁-₆haloalkyl, or C₃-₇cycloalkyl or in an alternative embodiment, aryl or aryl(alkyl)-.

In one embodiment, the process for the synthesis of the diastereomerically enriched Sₚ-phosphoramidate nucleotide of Formula XVI comprises:
(a) contacting a compound of Formula I with a compound of Formula XVII in the presence of an organolithium or organomagnesium reagent and optionally in the presence of an alkali halide to afford a protected diastereomerically enriched Sₚ-phosphoramidate nucleotide of Formula XVIII:

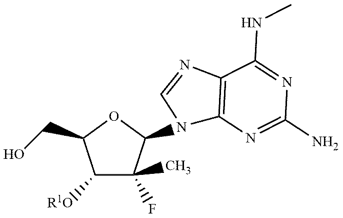

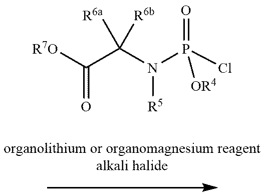

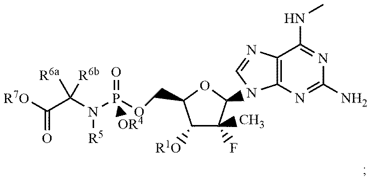

(b) further purifying the diastereomerically enriched Sₚ-phosphoramidate nucleotide of Formula XVIII to afford the diastereomerically pure Sₚ-purine phosphoramidate nucleotide of Formula XVIII wherein the diastereomeric purity is greater than about 90%, about 95%, or even about 99% or greater; and,
(c) deprotecting the protected phosphoramidate nucleotide of Formula XVIII to afford the diastereomerically pure Sₚ-phosphoramidate nucleotide of Formula XVI:

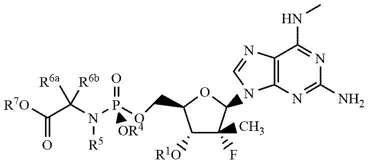

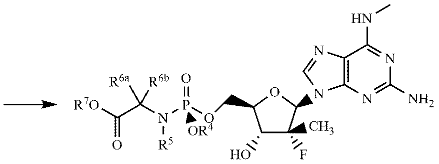

; and
(d) further optionally purifying the diastereomerically enriched compound of Formula XVI to afford the diastereomerically pure compound of Formula XVI wherein the diastereomeric purity is greater than about 90%, greater than about 95% or even greater than about 99% or greater; and,
(e) optionally converting the compound of Formula XVI to a pharmaceutically acceptable salt of a compound of Formula XVI;
wherein R², R⁴, R⁵, R^{6a}, R^{6b}, and R⁷ are as defined herein.

In an alternative embodiment, step comprises (a) contacting a compound of Formula I with a compound of Formula IA in the presence of an organolithium or organomagnesium reagent and optionally in the presence of an alkali halide to afford a protected diastereomerically enriched Sₚ-phosphoramidate nucleotide of Formula XVIII:

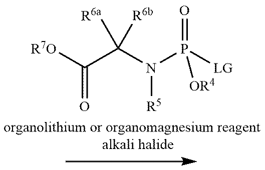

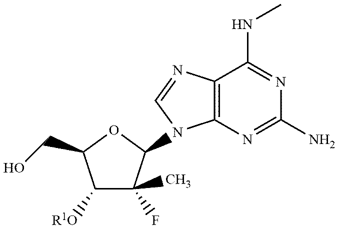

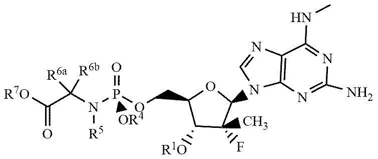

wherein
LG is a leaving group.

In certain embodiments, LG is selected from Br, I, tosylate, mesylate, trifluoroacetate, trifluorosulfonate, camphorsulfonate, triflate, acetate, —OSO₂R^{a}, aryloxide, and aryloxide substituted with at least one electron withdrawing group; and R^{a} is C₁₋₄alkyl, aryl, or aryl substituted with C₁₋₄alkyl, halogen, or nitro.

Non-limiting examples of electron withdrawing groups include, but are not limited to, halogen, NO₂, haloalkyl, —C(O)(alkyl), —C(O)(aryl), —C(O)O(alkyl), and —C(O)O(aryl).

Non-limiting examples of aryloxide groups substituted with at least one electron withdrawing group include p-nitrophenoxide, 2-chlorophenoxide, 4-chlorophenoxide, 2,4-dinitrophenoxide, and pentafluorophenoxide.

In an alternative embodiment, a compound of Formula IX, Formula XII, or Formula XIV is reacted with a compound of Formula XVII to afford a protected diastereomerically enriched Sₚ-phosphoramidate nucleotide that is then further purified and subsequently deprotected to afford a compound of Formula XVI.

In one embodiment, compounds with an alternative amino acid configuration are synthesized via the processes discussed above. The amino acid can be in the D- or L-configuration, or a mixture thereof, including a racemic mixture. The compound can be at least 51% free of the opposite D- or L-configuration, and can be at least 60%, 70%, 80%, 90%, 95%, 98%, 99% or even 100% free of the opposite D- or L-configuration. Non-limiting examples of compounds with alternative amino acid configurations include:

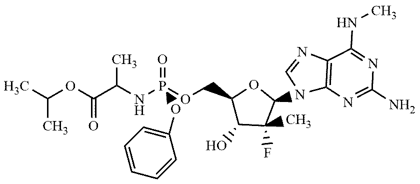 [SMILES: CNc1nc(N)nc2c1ncn2[C@@H]1O[C@H](CO[P@@](=O)(NC(C)C(=O)OC(C)C)Oc2ccccc2)[C@@H](O)[C@@]1(C)F]

or

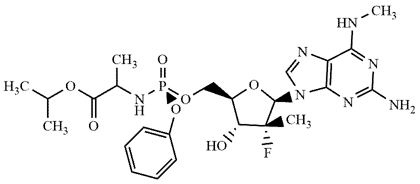 [SMILES: CNc1nc(N)nc2c1ncn2[C@@H]1O[C@H](CO[P@@](=O)(NC(C)C(=O)OC(C)C)Oc2ccccc2)[C@@H](O)[C@@]1(C)F]

Similarly, the present invention also provides processes for the pharmaceutically acceptable salts of compounds with alternative amino acid configurations, including the hemi-sulfate salt compounds:

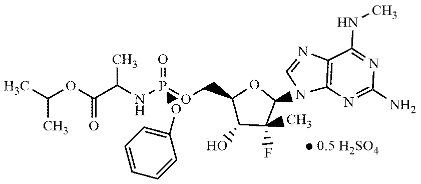

and

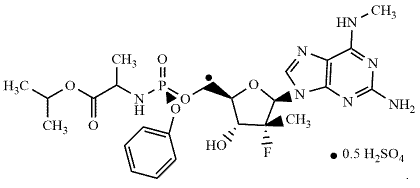

### DEFINITIONS

Compounds are described using standard nomenclature. Unless defined otherwise, all technical and scientific terms used herein have the same meaning as is commonly understood by one of skill in the art to which the invention belongs.

The terms “a” and “an” do not denote a limitation of quantity, but rather denote the presence of at least one of the referenced item. The term “or” means “and/or”. Recitation of ranges of values merely intend to serve as a shorthand method of referring individually to each separate value falling within the range, unless otherwise indicated herein, and each separate value is incorporated into the specification as if it were individually recited herein. The endpoints of all ranges are included within the range and independently combinable.

The term “amino acid” refers to a D- or L- natural or non-naturally occurring amino acid. Representative amino acids include, but are not limited to, alanine, β-alanine, arginine, asparagine, aspartic acid, cysteine, cystine, glutamic acid, glutamine, glycine, phenylalanine, histidine, isoleucine, lysine, leucine, methionine, proline, serine, threonine, valine, tryptophan, or tyrosine, among others.

All processes described herein can be performed in a suitable order unless otherwise indicated herein or otherwise clearly contradicted by context. The use of example, or exemplary language (for example, “such as”), is intended merely to better illustrate the invention and does not pose a limitation on the scope of the invention unless otherwise claimed.

Throughout the present application, the R/S system of nomenclature of enantiomers is followed. A chiral center having regard to the phosphorus atom P is labeled Rₚ or S_{P} according to a system in which the substituents on the atom P are each assigned a priority based on atomic number, according to the Cahn-Ingold-Prelog priority rules (CIP). Reference concerning the CIP rules is made to “Advanced Organic Chemistry” by J. March published by John Wiley & Sons (2007) and IUPAC Rules for the Nomenclature of Organic Chemistry, Section E, Stereochemistry (1974). The CIP rules allocate the lowest priority to the direct substituent on the chiral center P having the lowest atomic number. In the case of a phosphoramidate, this substituent is N. The P center is then orientated so that the N substituent is pointed away from the viewer. The atoms or next nearest atoms, if present, to the three O atoms directly linked to P are then considered, according to the CIP rules. If these atoms decrease in atomic number when viewed in a clockwise direction, the enantiomer is labeled R_{P}. If these atoms decrease in atomic number in a counterclockwise direction, the enantiomer is labeled S_{P}.

The symbol

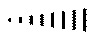

(dashed bond) present in some of the formulas of the specification and claims indicates that the substituent is directed below the plane of the sheet. The symbol

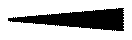

(wedge bond) present in some of the formulas of the specification and claims indicates that the substituent is directed above the plane of the sheet.

The compounds prepared by the processes of the present invention have one or more stereocenters, and may exist, be used or be isolated in diastereoisomerically pure forms or as diastereomeric enriched mixtures. It should be understood that the processes of the present invention may yield diastereoisomerically pure forms or diastereomeric enriched mixtures. It should also be understood that the products of the present invention may be isolated as diastereoisomerically pure forms or as diastereomeric enriched mixtures.

A diastereomeric mixture may contain the two diastereoisomers in any mutual ratio, unless otherwise indicated.

“Diastereomerically enriched” as used in the present application means that one of the diastereoisomers is present in excess of the other diastereoisomer.

“Diastereomerically pure” refers to a compound whose diastereoisomeric purity is at least about 90%, about 95%, or even about 99% or greater, and may be 100% pure. Alternatively, it may be at least about 80% or 85%-90% pure.

“Alkyl” is a branched or straight chain saturated aliphatic hydrocarbon group. In one non-limiting embodiment, the alkyl group contains from about 1 to about 6 carbon atoms, more generally from 1 to about 4 carbon atoms, or from 1 to about 3 carbon atoms. Examples of alkyl include, but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentance, 3-methylpentane, 2,2-dimethylbutane, and 2,3-dimethylbutane. Alkyl can include cycloalkyl.

“Cycloalkyl” is a saturated group containing all carbon rings and from 3 to 6 carbon atoms (“C₃-C₆cycloalkyl”) and zero heteroatoms in a monocyclic or polycyclic (for example, bicyclic or tricyclic) non-aromatic ring system. Non-limiting examples of “cycloalkyl” include: cyclopropyl, cyclobutyl, cyclopentyl, and cyclohexyl.

Any compound used in or formed by the processes described herein may be modified to make an inorganic or organic acid or base addition salt thereof to form a “pharmaceutically acceptable salt”, if appropriate and desired. The salts of the present compounds can be prepared from a parent compound that contains a basic or acidic moiety by chemical processes. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds may optionally be provided in the form of a solvate.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the salts and the quaternary ammonium salts of the parent compound formed, for example, from inorganic or organic acids that are not unduly toxic. For example, acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH2)n—COOH where n is 0-4, and the like, or using a different acid that produces the same counterion. Lists of additional suitable salts may be found, for example, in Remington’s Pharmaceutical Sciences, 17th ed., Mack Publishing Company, Easton, Pa., p. 1418 (1985).

The term C₁ to C₈ alcohol refers to a straight/branched and/or cyclic/acyclic alcohol having any of the number of carbons within the range, and the range is specifically intended to independently disclose each compound within the range. The C₁ to C₈ alcohol includes, but is not limited to, methanol, ethanol, n-propanol, isopropanol, isobutanol, hexanol, and cyclohexanol.

The term C₂ to C₈ ether refers to a straight/branched and/or cyclic/acyclic ether having any of the number of carbons within the range, and the range is specifically intended to independently disclose each compound within the range. The C₂ to C₈ ether includes, but is not limited to, dimethyl ether, diethyl ether, di-isopropyl ether, di-n-butyl ether, methyl-t-butyl ether (MTBE), tetrahydrofuran, and dioxane

The term C₃ to C₇ ketone refers to a straight/branched and/or cyclic/acyclic ketone having any of the number of carbons within the range, and the range is specifically intended to independently disclose each compound within the range. The C₃ to C₇ ketone includes, but is not limited to, acetone, methyl ethyl ketone, propanone, butanone, methyl isobutyl ketone, methyl butyl ketone, and cyclohexanone.

The term C₃ to C₇ ester refers to a straight/branched and/or cyclic/acyclic ester having any of the number of carbons within the range, and the range is specifically intended to independently disclose each compound within the range. The C₃ to C₇ ester includes, but is not limited to, ethyl acetate, propyl acetate, n-butyl acetate, etc.

The term C₁ to C₂ chlorocarbon refers to a chlorocarbon with 1 or 2 carbons, with any number of chloro atoms that fulfill the desired purpose. The C₁ to C₂ chlorocarbon includes, but is not limited to, chloroform, methylene chloride (DCM), carbon tetrachloride, 1,2-dichloroethane, and tetrachloroethane.

A term C₂ to C₇ nitrile refers to a nitrile having any of the number of carbons within the range, and the range is specifically intended to independently disclose each compound within the range. The C₂ to C₇ nitrile includes, but is not limited to, acetonitrile, propionitrile, etc.

Alternative solvents include, but are not limited to, diethylene glycol, diglyme (diethylene glycol dimethyl ether), 1,2-dimethoxy-ethane, dimethylformamide, dimethylsulfoxide, ethylene glycol, glycerin, hexamethylphsphoramide, hexamethylphosphorous triame, N-methyl-2-pyrrolidinone, nitromethane, pyridine, triethyl amine, and acetic acid.

The term C₅ to C₁₂ saturated hydrocarbon refers to a straight/branched and/or cyclic/acyclic hydrocarbon having any of the number of carbons within the range, and the range is specifically intended to independently disclose each compound within the range. The C₅ to C₁₂ saturated hydrocarbon includes, but is not limited to, pentane (including n-pentane), petroleum ether (ligroine), hexane (including n-hexane), heptane (including n-heptane), cyclohexane, and cycloheptane.

The term C₆ to C₁₂ aromatic refers to a phenyl group or a substituted or unsubstituted hydrocarbon having a phenyl group in its backbone. Examples of C₆ to C₁₂ aromatics include benzene, xylene, toluene, chlorobenzene, o-xylene, m-xylene, p-xylene, xylenes, aniline, nitrobenzene, and benzyl alcohol. Alternatively, the C₆ to C₁₂ aromatic refers to a heteroaryl or a substituted or unsubstituted hydrocarbon having a heteroaryl in its backbone, for example pyridine. Alternatively, the C₆ to C₁₂ aromatic can also include a fused aryl group or biphenyl group.

The term “leaving group” has the same meaning to the skilled artisan (Advanced Organic Chemistry: reactions, mechanisms and structure-Fourth Edition by Jerry March, John Wiley and Sons Ed.; 1992 pages 351-357) and represents a group which is part of and attached to a substrate molecule. In a reaction where the substrate molecule undergoes a displacement reaction (with for example a compound of Formula I), the leaving group is then displaced.

### Nucleoside Compounds of Formula I, Formula IX, Formula XII, and Formula XIV

In certain aspects of the present invention, the synthesis of Compound 1 comprises the first step of synthesizing a nucleoside compound of Formula I:

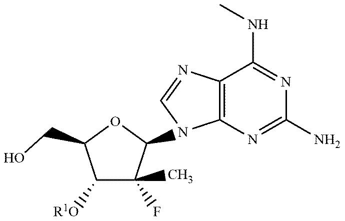

wherein R¹ is an oxygen protecting group which when attached to the oxygen is a moiety selected from a substituted benzyl ether, 4-bromobenzoate, p-methoxybenzyloxymethyl ether (MPBM), o-nitrobenzyloxymethyl ether (NBOM), p-nitrobenzyloxymethyl ether, t-butoxymethyl ether, 2,2,2-trichloroethoxymethyl ether, 3-bromotetrahydropyranyl ether, tetrahydropyranyl ether, tetrahydrothiopyranyl ether, 1-methoxycyclohexyl ether, 1,4-dioxan-2-yl ether, tetrahydrofuranyl ether, tetrahydrothiofuranyl ether, a substituted phenyl ester, 2-picolyl ether, 4-picolyl ether, 1,3-benzodithiolan-2-yl ether, p-chlorophenoxyacetate ester, 3-phenylpropionate ester, p-phenylbenzoate ester, alkyl p-nitrophenyl carbonyl, alkyl benzyl carbonyl, alkyl p-methoxybenzyl carbonyl, alkyl o-nitrobenzyl carbonyl, alkyl p-nitrobenzyl carbonyl, t-butylcarbonyl (Boc), and benzylcarbonyl (Cbz); and
wherein the substituent is selected from alkoxy, hydroxy, nitro, bromo, chloro, fluoro, azido, and haloalkyl.

Non-limiting examples of R¹ include:

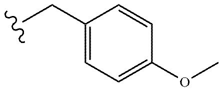

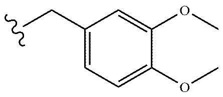

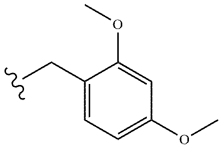

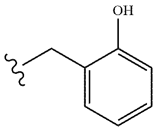

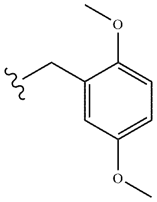

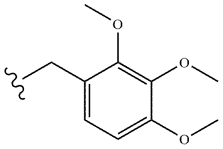

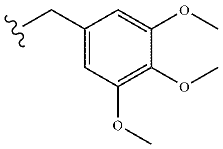

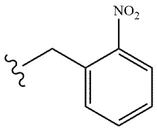

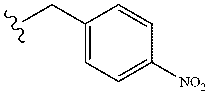

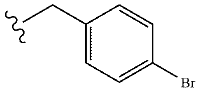

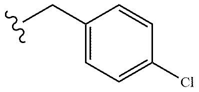

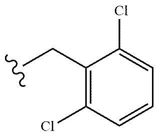

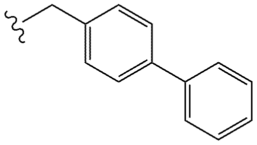

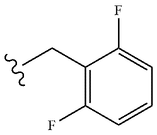

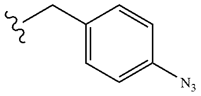

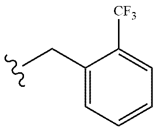

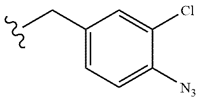

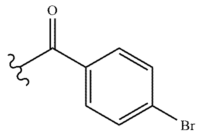

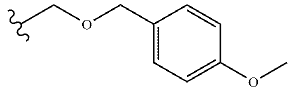

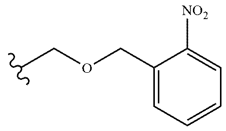

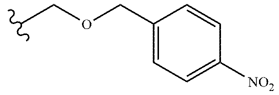

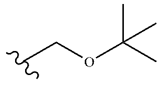

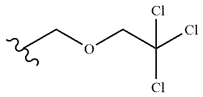

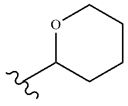

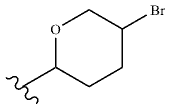

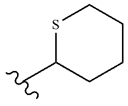

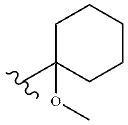

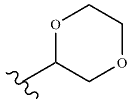

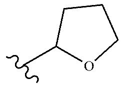

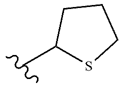

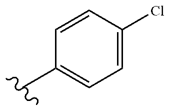

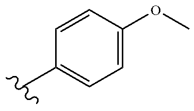

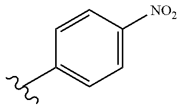

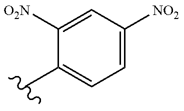

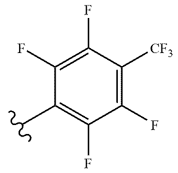

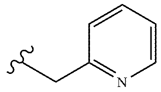

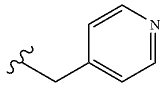

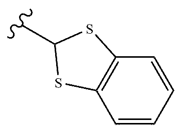

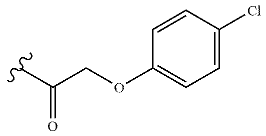

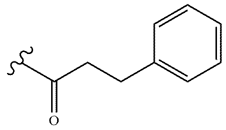

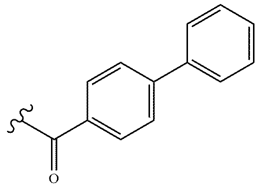

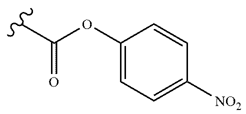

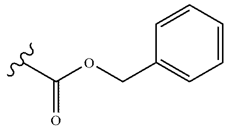

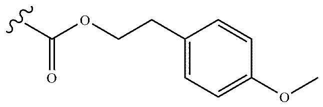

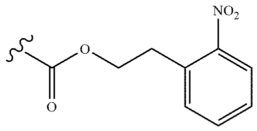

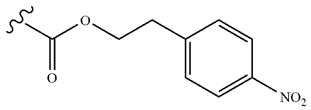

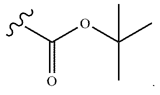

In certain embodiments, the synthesis of Formula I comprises the steps of:
(l.a) the selective protection of the primary 5' hydroxyl group on the nucleoside (3S,4R,5R)-3,4-dihydroxy-5-(hydroxymethyl)-3-methyldihydrofuran-2(3H)-one to afford a compound of Formula III wherein R² is an oxygen protecting group which when attached to the oxygen is an ester, ether, or silyl ether moiety:

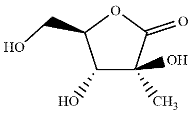

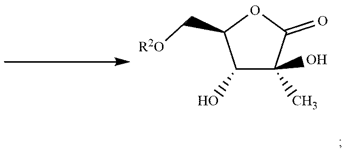

(1.b) the protection of the 3’-OH group of the nucleoside compound of Formula III with an oxygen protecting group R¹ to afford a nucleoside compound of Formula VI wherein the oxygen protecting group, R¹, assists in the direction of stereoselective addition of the phosphoramidate toward the Sₚ- diastereomer:

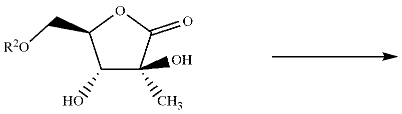

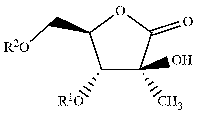

(1.c) the conversion of the alcohol of Formula IV into a monofluoride with inversion of stereochemistry to afford a compound of Formula V:

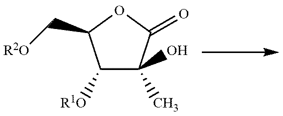

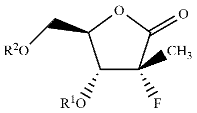

(1.d) the reduction of the lactone of the nucleoside compound of Formula V to afford the nucleoside compound of Formula VI:

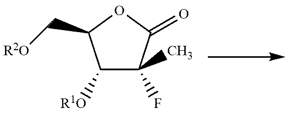

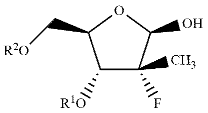

(1.e) the conversion of a compound of Formula VI to a compound of Formula VII wherein X is C1, Br, or OAc:

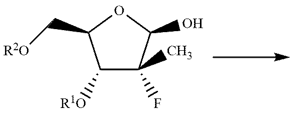

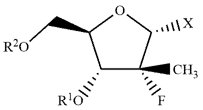

(1.f) the nucleophilic substitution of the compound of Formula VII with 2-amino-6-chloropurine to afford a compound of Formula VIII:

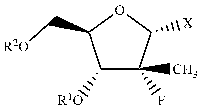

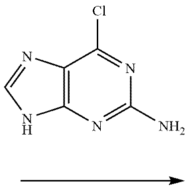 [SMILES: Nc1nc(Cl)c2nc[nH]c2n1]

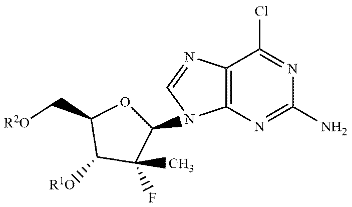

; and
(1.g) the conversion of the 2-amino-6-chloropurine base to the 2-amino-N⁶-methyl base and the selective deprotection of the 5’-position to afford a compound of Formula I:

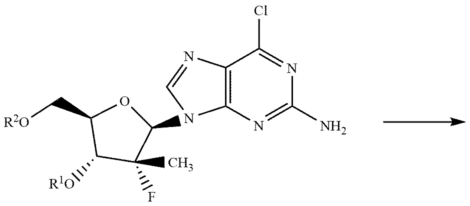

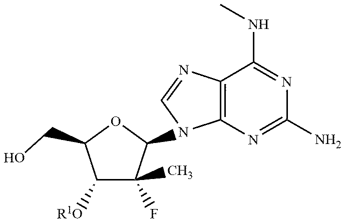

In certain aspects of the present invention, the synthesis of Compound 1 includes the first step of synthesizing a nucleoside compound of Formula IX:

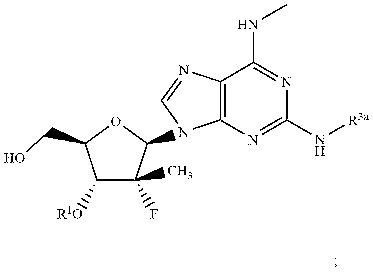

wherein R¹ and R^{3a} are as defined herein.

In certain embodiments, the synthesis of a compound of Formula IX comprises the steps:
(1.f.2) protecting the N²-position in the compound of Formula VIII with protecting group R^{3a} to afford a compound of Formula XI wherein R^{3a} is nitrogen protecting group which when attached to the nitrogen is an amine, amide, or carbamate moiety:

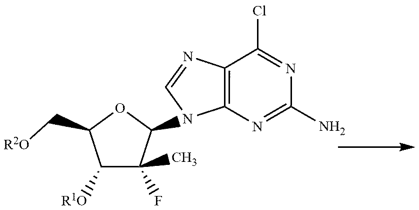

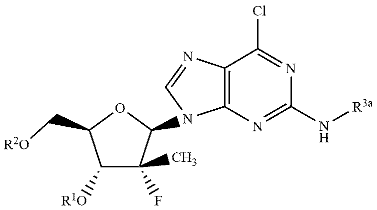

; and
(1.g) converting the 6-chloro position in the compound of Formula XI to the N⁶-methylamino group and selectively deprotecting the R¹ position to afford a compound of Formula IX:

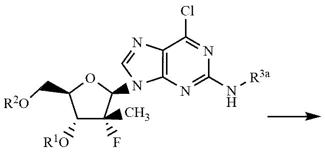

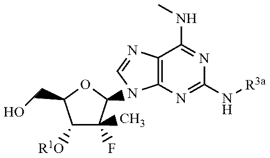

In an alternative embodiment, the preparation of a compound of Formula IX includes the nucleophilic substitution reaction of Formula VII with 2-amino-6-chloropurine wherein the 2-amino is protected with R^{3a} to afford a compound of Formula IX' that is then converted to a compound of Formula IX:

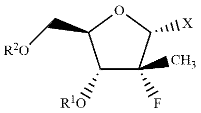

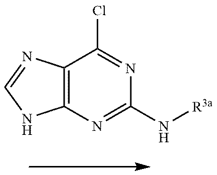

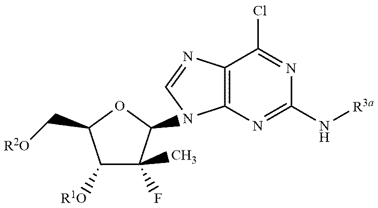

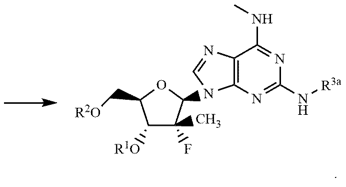

In one aspect of the present invention, the synthesis of Compound 1 comprises the first step of synthesizing a nucleoside compound of Formula XII:

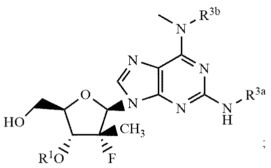

wherein R¹, R^{3a}, and R^{3b} are as defined herein.

In one embodiment, the synthesis of a compound of Formula XII comprises protecting the N⁶-methylamine in the compound of Formula IX with protecting group R^{3b}to afford a compound of Formula XII wherein R^{3b} is a nitrogen protecting group which when attached to the nitrogen is an amine, amide, or carbamate moiety:

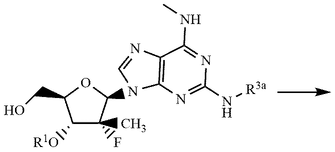

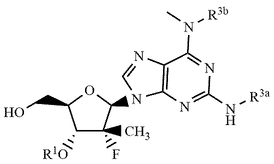

In an alternative embodiment, the synthesis of a compound of Formula XII comprises the nucleophilic substitution reaction of Formula VII with 2-amino-N⁶-methylaminopurine wherein the 2-amino and the N⁶-methylamino are protected with to afford a compound of Formula XII’ that is then converted to a compound of Formula XII:

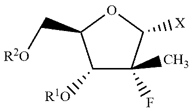

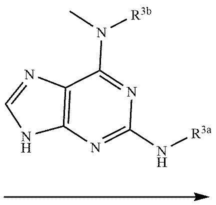

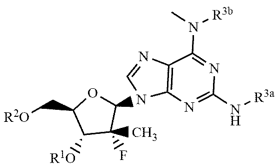

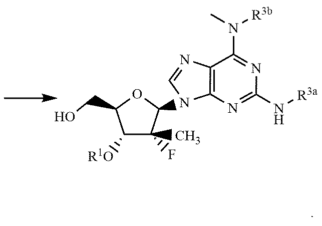

In one aspect of the present invention, the synthesis of Compound 1 comprises the first step of synthesizing a nucleoside compound of Formula XIV:

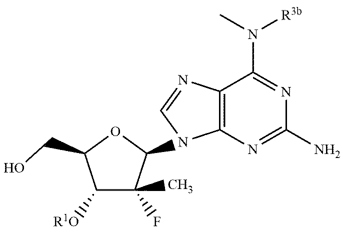

wherein R¹ and R^{3b} are as defined herein.

In one embodiment, a compound of Formula XIV is synthesized by protecting the N⁶-methylamine in the compound of Formula I with protecting group R^{3b} to afford a compound of Formula XIV where the N⁶-methylamine positions:

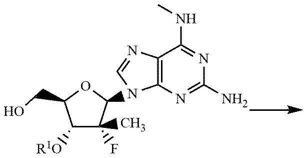

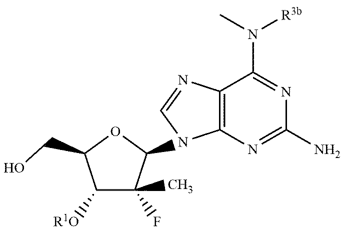

In an alternative embodiment, the synthesis of a compound of Formula XIV is conducted by selectively deprotecting the N²-position of a compound of Formula XII:

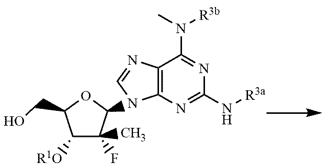

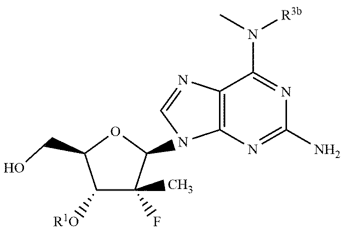

In an alternative embodiment of the present invention, the synthesis of a compound of Formula I comprises the steps of:
(1.a.2) the selective protection of the primary 5’-hydroxyl group on the nucleoside (3R,4R,5R)-3-fluoro-4-hydroxy-5-(hydroxymethyl)-3-methyldihydrofuran-2(3H)-one to afford a compound of Formula XIX wherein R² is an oxygen protecting group which when attached to the oxygen is an ester, ether, or silyl ether moiety:

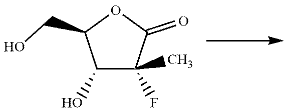

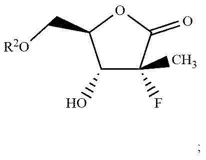

(1.b.2) the protection of the 3'-OH group of the nucleoside compound of Formula XIX with an oxygen protecting group R¹ to afford a nucleoside compound of Formula V wherein the oxygen protecting group, R¹, assists in the direction of stereoselective addition of the phosphoramidate toward the Sₚ- diastereomer:

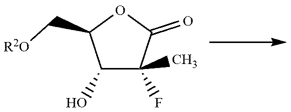

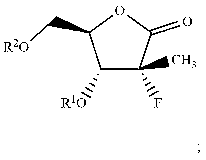

(1.c.2) the reduction of the lactone of the nucleoside compound of Formula V to afford the nucleoside compound of Formula VI:

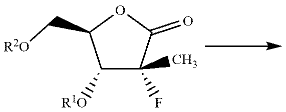

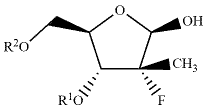

(1.d.2) the conversion of a compound of Formula VI to a compound of Formula VII wherein X is C1, Br, or OAc:

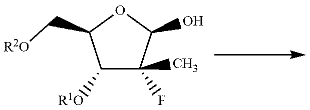

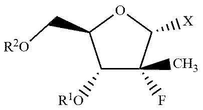

(1.e.2) the nucleophilic substitution of the compound of Formula VII with 2-amino-6-chloropurine to afford a compound of Formula VIII:

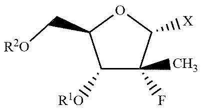

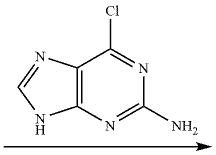 [SMILES: Nc1nc(Cl)c2nc[nH]c2n1]

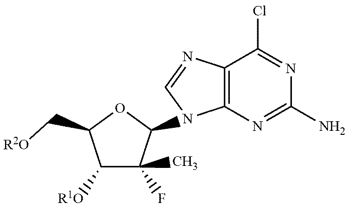

; and (1.f.1.2) the conversion of the 2-amino-6-chloropurine base to the 2-amino-N⁶-methyl base and the selective deprotection of the 5’-position to afford a compound of Formula I:

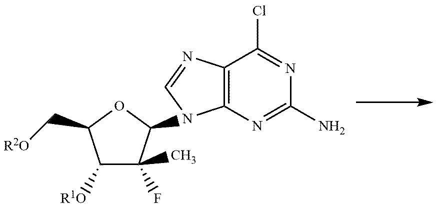

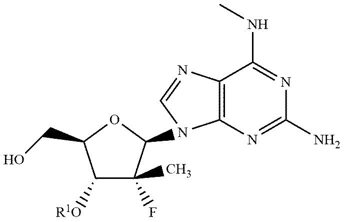

Non-limiting examples of nucleoside compounds of Formula I include:

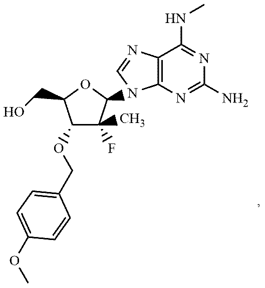

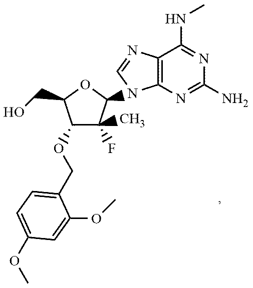

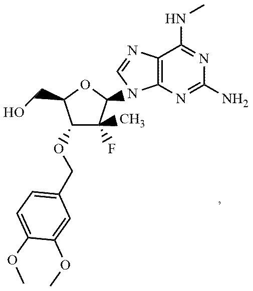

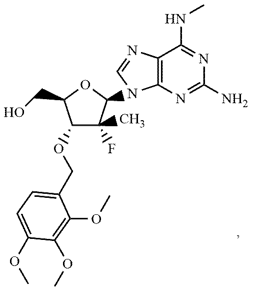

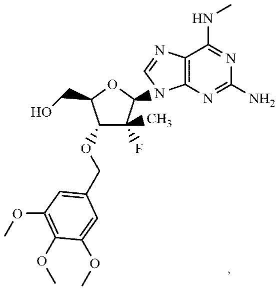

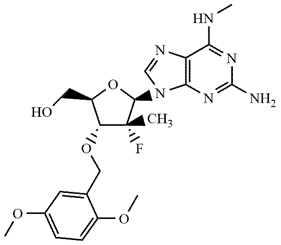 [SMILES: CNc1nc(N)nc2c1ncn2[C@@H]1O[C@H](CO)[C@@H](OCc2cc(OC)ccc2OC)[C@@]1(C)F]

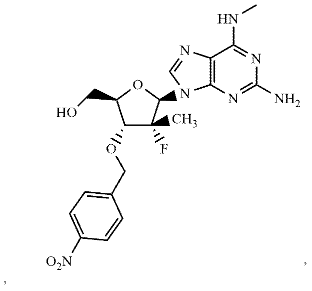

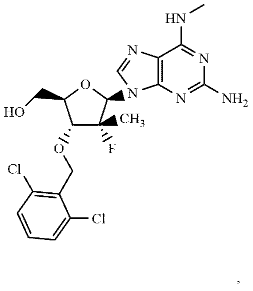

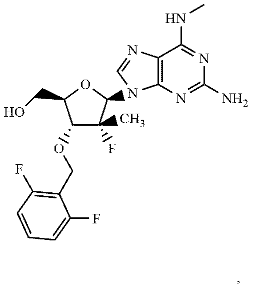

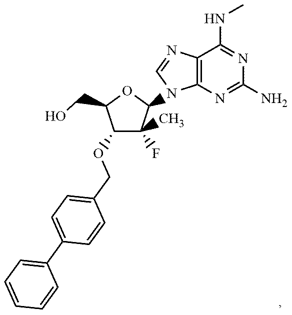

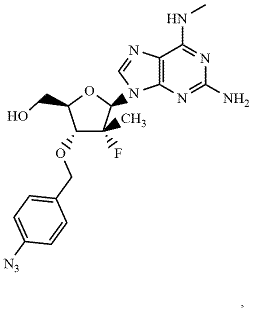

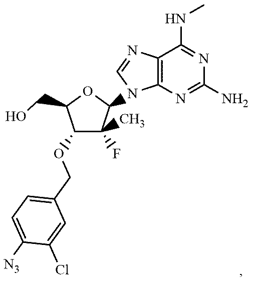

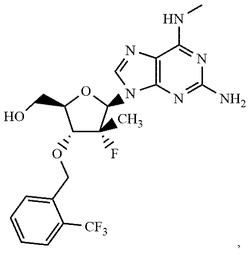

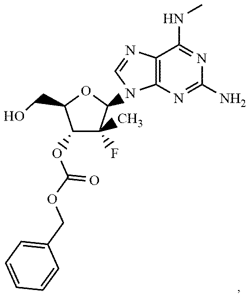

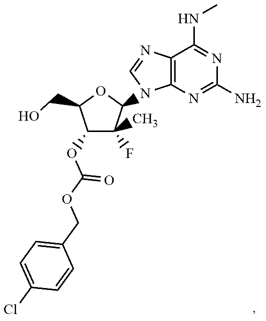

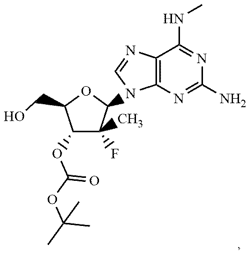

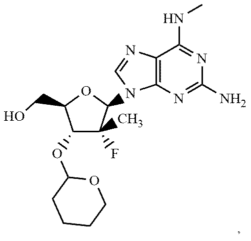

Non-limiting examples of nucleoside compounds of Formula IX include:

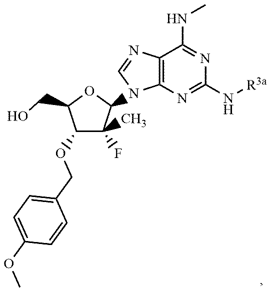

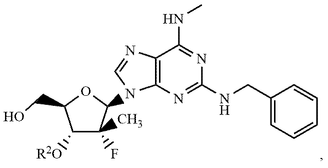

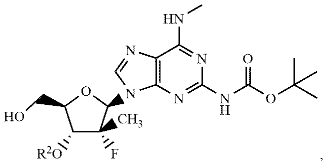

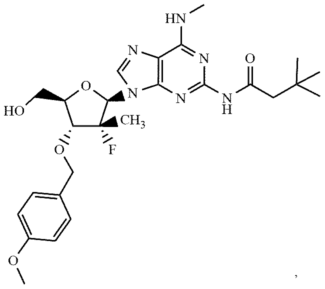

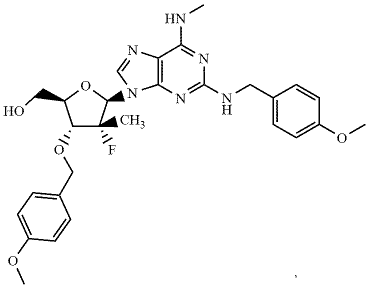

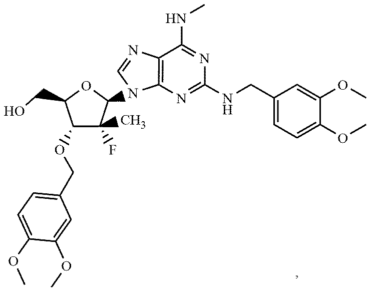

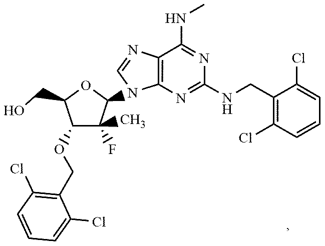

and

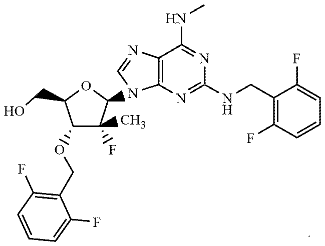

Non-limiting examples of nucleoside compounds of Formula XII include:

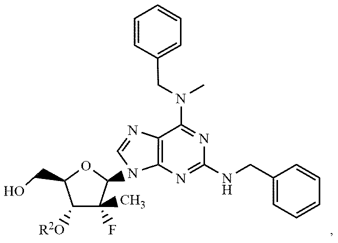

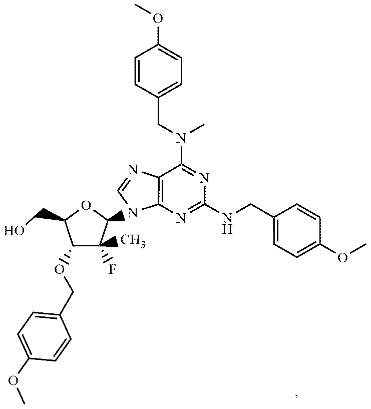

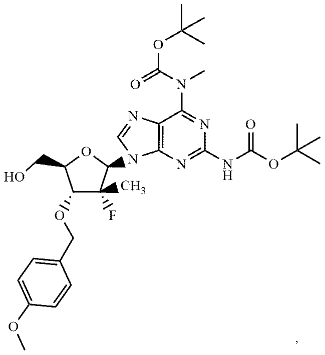

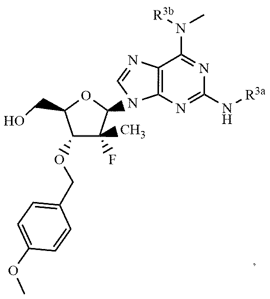

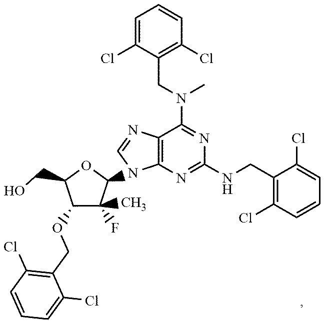

and

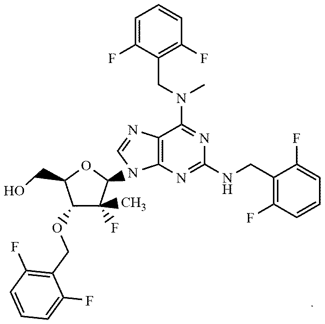

Non-limiting examples of nucleoside compounds of Formula XIV include:

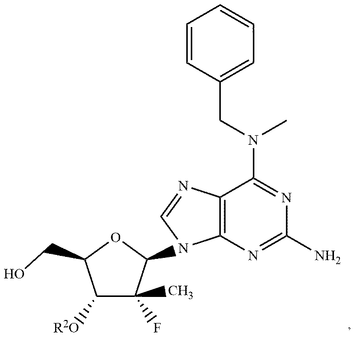

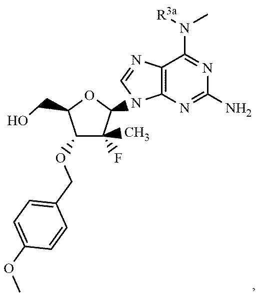

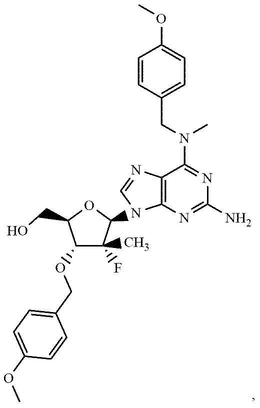

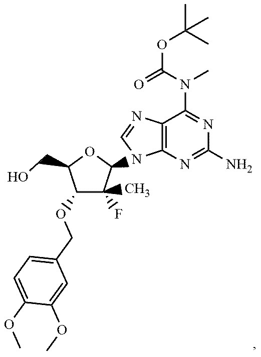

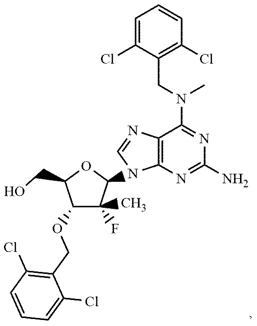

and

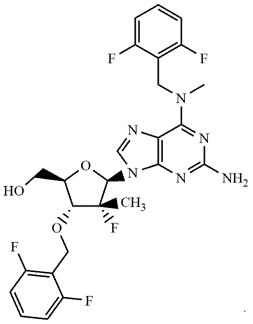

### Nucleophilic Substitution of Isopropyl (chloro(phenoxy)phosphoryl)-L-alaninate or Intermediate I with a Nucleoside Compound of Formula I, Formula IX, Formula XII, or Formula XIV and Subsequent Deprotection

In one aspect of the present invention, the process for synthesizing the diastereomerically pure Sₚ-phosphoramidate nucleotide of Compound 1 comprises the steps of:
(a) contacting the nucleoside compound of Formula I with isopropyl (chloro(phenoxy)phosphoryl)-Z-alaninate in the presence of an organolithium or organomagnesium reagent and optionally in the presence of an alkali halide to afford a protected diastereomerically enriched Sₚ-phosphoramidate nucleotide of Formula II wherein the Sₚ-diastereomer is in excess of the Rₚ-diastereomer:

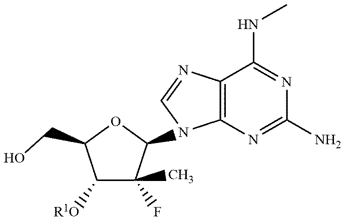

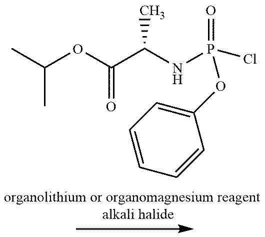

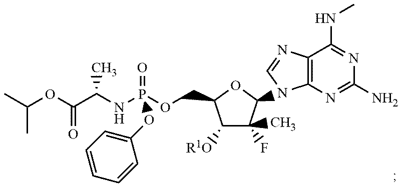

(b) further optionally purifying, for example, by selective crystallization, the diastereomerically enriched Sₚ-phosphoramidate nucleotide of Formula II to afford the diastereomerically pure Sₚ-purine phosphoramidate nucleotide of Formula II wherein the diastereomeric purity is greater than about 90%, about 95% or even greater than about 99% or more; and,
(c) deprotecting the protected phosphoramidate nucleotide of Formula II to afford the diastereomerically pure Sₚ-phosphoramidate nucleotide Compound 1:

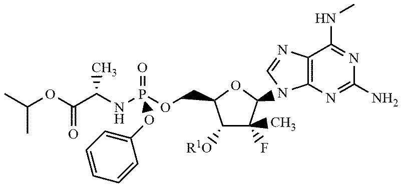

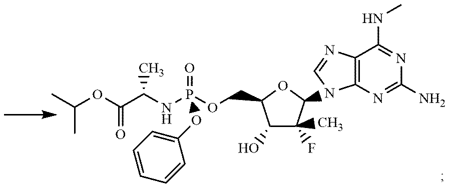

wherein:

R¹ is an oxygen protecting group which when attached to the oxygen is a moiety selected from a substituted benzyl ether, 4-bromobenzoate, p-methoxybenzyloxymethyl ether (MPBM), o-nitrobenzyloxymethyl ether (NBOM), p-nitrobenzyloxymethyl ether, t-butoxymethyl ether, 2,2,2-trichloroethoxymethyl ether, 3-bromotetrahydropyranyl ether, tetrahydropyranyl ether, tetrahydrothiopyranyl ether, 1-methoxycyclohexyl ether, 1,4-dioxan-2-yl ether, tetrahydrofuranyl ether, tetrahydrothiofuranyl ether, a substituted phenyl ether, 2-picolyl ether, 4-picolyl ether, 1,3-benzodithiolan-2-yl ether, p-chlorophenoxyacetate ester, 3-phenylpropionate ester, p-phenylbenzoate ester, alkyl p-nitrophenyl carbonyl, alkyl benzyl carbonyl, alkyl p-methoxybenzyl carbonyl, alkyl o-nitrobenzyl carbonyl, alkyl p-nitrobenzyl carbonyl, t-butylcarbonyl (Boc), and benzylcarbonyl (Cbz); and

wherein the substituent is selected from alkoxy, hydroxy, nitro, bromo, chloro, fluoro, azido, and haloalkyl.

In an alternative embodiment, step (a) comprises contacting the nucleoside compound of Formula I with Intermediate I in the presence of an organolithium or organomagnesium reagent and optionally in the presence of an alkali halide to afford a protected diastereomerically enriched Sₚ-phosphoramidate nucleotide of Formula II wherein the Sₚ-diastereomer is in excess of the Rₚ-diastereomer:

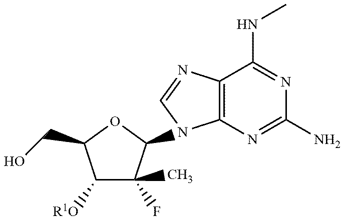

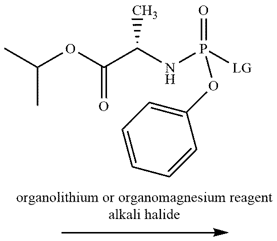

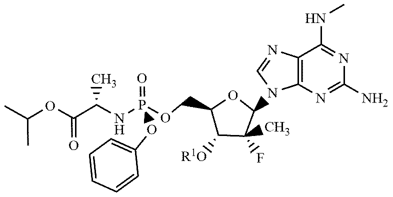

wherein LG is a leaving group.

In certain embodiments, LG is selected from Br, I, tosylate, mesylate, trifluoroacetate, trifluorosulfonate, camphorsulfonate, triflate, acetate, —OSO₂R^{a}, aryloxide, and aryloxide substituted with at least one electron withdrawing group; and

R^{a} is C₁₋₄alkyl, aryl, or aryl substituted with C₁₋₄alkyl, halogen, or nitro.

Non-limiting examples of electron withdrawing groups include, but are not limited to, halogen, NO₂, haloalkyl, —C(O)(alkyl), —C(O)(aryl), —C(O)O(alkyl), and —C(O)O(aryl).

Non-limiting examples of aryloxide groups substituted with at least one electron withdrawing group include p-nitrophenoxide, 2-chlorophenoxide, 4-chlorophenoxide, 2,4-dinitrophenoxide, and pentafluorophenoxide,

In an alternative aspect, the stereoselective synthesis of Compound 1 comprises:
(a) contacting the nucleoside compound of Formula IX with isopropyl (chloro(phenoxy)phosphoryl)-Z-alaninate in the presence of an organolithium or organomagnesium reagent and optionally in the presence of an alkali halide to afford a protected diastereomerically enriched Sₚ-phosphoramidate nucleotide of Formula X wherein the Sₚ-diastereomer is in excess of the Rₚ-diastereomer:

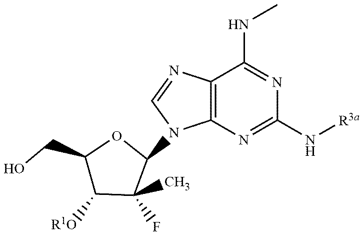

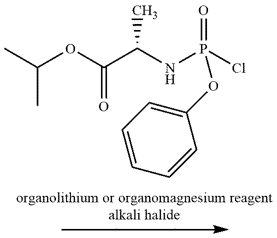

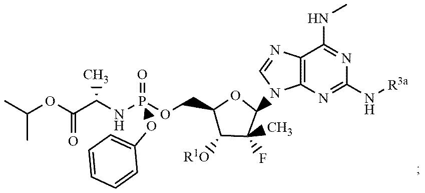

(b) further optionally purifying, for example, by selective crystallization, the diastereomerically enriched Sₚ-phosphoramidate nucleotide of Formula X to afford the diastereomerically pure Sₚ-purine phosphoramidate nucleotide of Formula X wherein the diastereomeric purity is greater than about 90%, about 95% or even about 99% or greater; and, (c) deprotecting the protected phosphoramidate nucleotide of Formula X to afford the diastereomerically pure Sₚ-phosphoramidate nucleotide Compound 1:

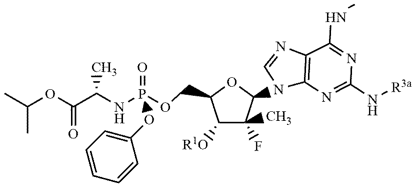

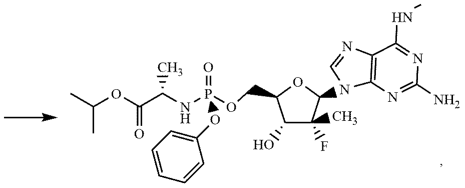

wherein:

R³ is a nitrogen protecting group which when attached to the nitrogen is a moiety selected from an amine, amide, or carbamate moiety for example, a benzyl amine.

Non-limiting examples of carbamate protecting groups include methyl and ethyl carbamate, t-butyl (BOC) carbamate, benzyl carbamate (CBz). The installation of these protecting groups can be conducted according to a procedure described in Theodora W. Green, Protective Groups in Organic Synthesis, Third Edition, John Wiley & Sons (1999), for example on pages 504-537. Non-limiting examples of amine protecting groups include methyl, t-butyl, allyl, benzyl, 4-methoxybenzyl, 2,4-dimethoxybenzyl, and 2-hydroxybenzyl. The installation of these protecting groups can be conducted according to one of the procedures described in Theodora W. Green, Protective Groups in Organic Synthesis, Third Edition, John Wiley & Sons (1999), which is incorporated by reference, for example on pages 573-586.

In an alternative embodiment, step (a) comprises contacting the nucleoside compound of Formula IX with Intermediate I in the presence of an organolithium or organomagnesium reagent and optionally in the presence of an alkali halide to afford a protected diastereomerically enriched Sₚ-phosphoramidate nucleotide of Formula X wherein the Sₚ-diastereomer is in excess of the Rₚ-diastereomer:

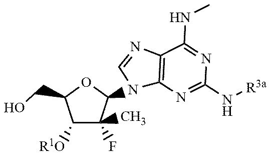

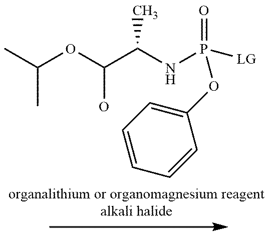

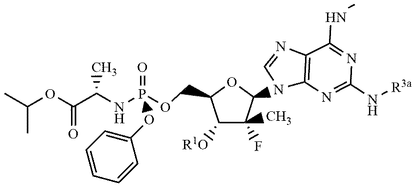

wherein LG, R¹, and R^{3a} are as defined herein.

In a further alternative aspect, the stereoselective synthesis of Compound 1 comprises:
(a) contacting the nucleoside compound of Formula XII with isopropyl (chloro(phenoxy)phosphoryl)-Z-alaninate in the presence of an organolithium or organomagnesium reagent and optionally in the presence of an alkali halide to afford a protected diastereomerically enriched Sₚ-phosphoramidate nucleotide of Formula XIII wherein the Sₚ-diastereomer is in excess of the Rₚ-diastereomer:

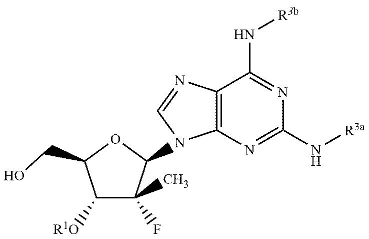

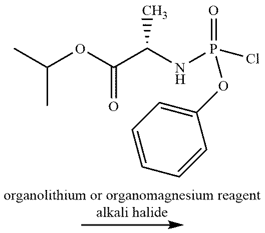

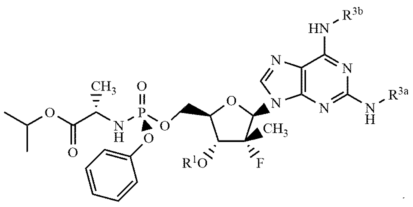

(b) further optionally purifying, for example, by selective crystallization, the diastereomerically enriched Sₚ-phosphoramidate nucleotide of Formula XIII to afford the diastereomerically pure Sₚ-purine phosphoramidate nucleotide of Formula XIII wherein the diastereomeric purity is greater than about 90%, about 95% or even about 99% or greater; and,
(c) deprotecting the protected phosphoramidate nucleotide of Formula XIII to afford the diastereomerically pure Sₚ-phosphoramidate nucleotide Compound 1:

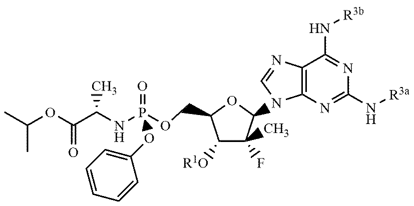

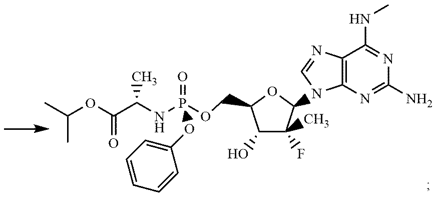

wherein R¹, R^{3a}, and R^{3b} are defined herein.

In an alternative embodiment, step (a) comprises contacting the nucleoside compound of Formula XII with Intermediate I in the presence of an organolithium or organomagnesium reagent and optionally in the presence of an alkali halide to afford a protected diastereomerically enriched Sₚ-phosphoramidate nucleotide of Formula XIII wherein the Sₚ-diastereomer is in excess of the Rₚ-diastereomer:

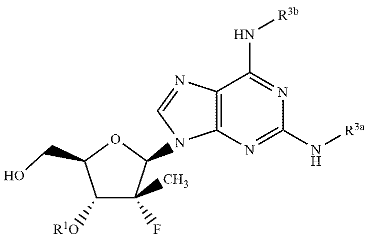

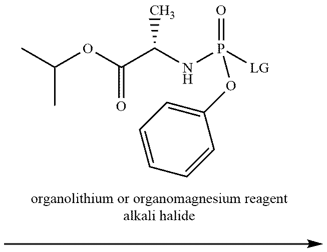

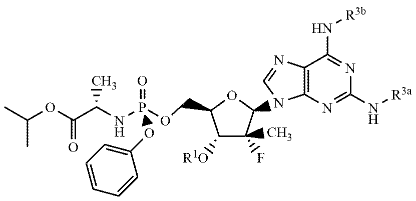

wherein LG, R¹, R^{3a}, and R^{3b} are as defined herein.

In a further alternative aspect, the stereoselective synthesis of Compound 1 includes:
(a) contacting the nucleoside compound of Formula XIV with isopropyl (chloro(phenoxy)phosphoryl)-Z-alaninate in the presence of an organolithium or organomagnesium reagent and optionally in the presence of an alkali halide to afford a protected diastereomerically enriched Sₚ-phosphoramidate nucleotide of Formula XV wherein the Sₚ-diastereomer is in excess of the Rₚ-diastereomer:

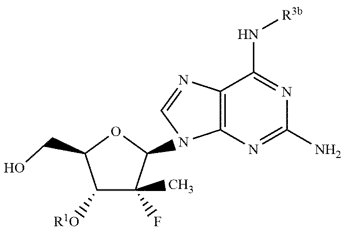

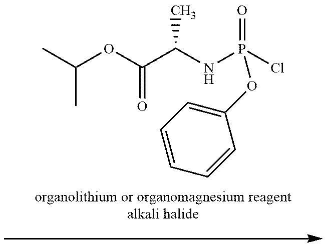

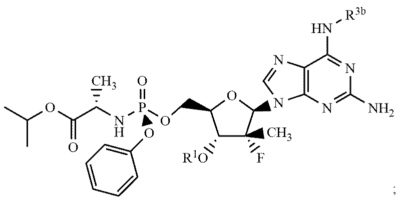

(b) further optionally purifying, for example, by selective crystallization, the diastereomerically enriched Sₚ-phosphoramidate nucleotide of Formula XV to afford the diastereomerically pure Sₚ-purine phosphoramidate nucleotide of Formula XV wherein the diastereomeric purity is greater than about 90%, about 95% or even about 99% or greater; and,
(c) deprotecting the protected phosphoramidate nucleotide of Formula XV to afford the diastereomerically pure Sₚ-phosphoramidate nucleotide Compound 1:

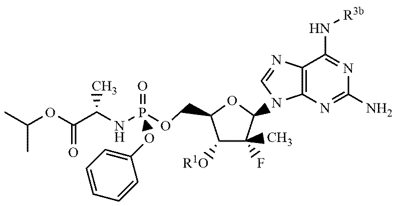

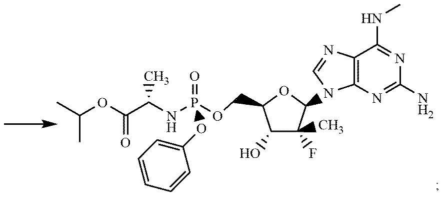

wherein R¹, R^{3a}, and R^{3b} are defined herein.

In an alternative embodiment, step (a) comprises contacting the nucleoside compound of Formula XIV with Intermediate I in the presence of an organolithium or organomagnesium reagent and optionally in the presence of an alkali halide to afford a protected diastereomerically enriched Sₚ-phosphoramidate nucleotide of Formula XV wherein the Sₚ-diastereomer is in excess of the Rₚ-diastereomer:

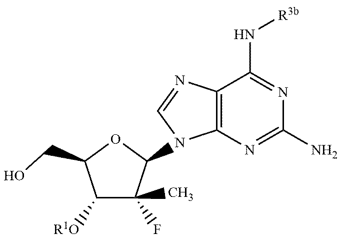

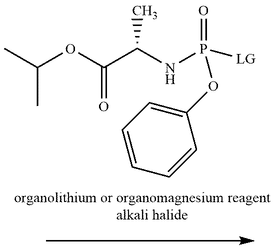

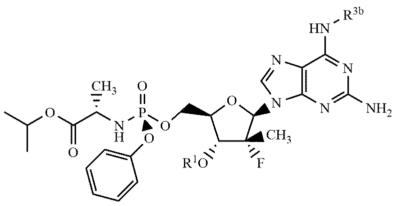

wherein LG, R¹, and R^{3b} are as defined herein.

The nucleophilic substitution of isopropyl (chloro(phenoxy)phosphoryl)-Z-alaninate or Intermediate I with a nucleoside compound of Formula I, Formula IX, Formula XII, or Formula XIV involves treatment of a nucleoside compound of Formula I, Formula IX, Formula XII, or Formula XIV with isopropyl (chloro(phenoxy)phosphoryl)-Z-alaninate or Intermediate I, optionally solubilized in an alkyl ether (for example, methyl tert-butyl ether) and with an organolithium reagent, an organomagnesium reagent or a mixture thereof, for example, at a temperature of between -60 and 0° C., for example between -40 and -20° C., in an ether, for example, tetrahydrofuran.

Non-limiting examples of organolithium reagents of the present invention are, for example, sec-butyllithium or tert-butyllithium. Non-limiting examples of organomagnesium reagents of the present invention, are for example, a tert-butyl halide or iso-propylmagnesium, including tert-butyl or iso-propylmagnesium chloride.

In an alternative embodiment, a lithium, sodium, potassium or magnesium amides, for example, sodium hexamethyldisilazide (NaHMDS), lithium (LiHMDS) or potassium (KHMDS), lithium diisopropylamide (LDA) or magnesium bis(diisopropylamide) (MDA) are used in the nucleophilic substitution reaction instead of an organolithium reagent, an organomagnesium reagent or a mixture thereof.

If the nucleophilic substitution reaction is conducted with an organomagnesium reagent, it is sometimes useful to also conduct the reaction in the presence of an alkali halide (for example a chloride), for example a lithium, sodium, potassium or cesium chloride. In an alternative embodiment, the reaction is conducted in the presence of a zinc or copper halide.

The amount of isopropyl (chloro(phenoxy)phosphoryl)-Z-alaninate or Intermediate I used in the nucleophilic substitution reaction in some embodiments ranges between about 1 and 4 equivalents with respect to the amount of the nucleoside compound of Formula I, Formula IX, Formula XII, or Formula XIV used, and for example, between 2 and 3 equivalents.

The amount of organolithium reagent, organomagnesium reagent or of the mixture thereof used in the nucleophilic substitution reaction in certain embodiments ranges between about 1 and 4 equivalents with respect to the amount of the nucleoside compound of Formula I, Formula IX, Formula XII, or Formula XIV used, for example between about 2 and 3 equivalents.

The amount of lithium, sodium, potassium or magnesium amide used in the nucleophilic substitution reaction in certain embodiments ranges between about 1 and 3 equivalents with respect to the amount of the nucleoside compound of Formula I, Formula IX, Formula XII, or Formula XIV used, for example, about 2 equivalents.

The alkaline, zinc or copper halide is used in certain embodiments in an amount ranging between about 0.5 and 2 equivalents with respect to the amount of organomagnesium used, and for example, about 1 equivalent.

In one aspect, the nucleophilic substitution reaction is conducted in a mixture of ethers, for example consisting of tetrahydrofuran and methyl tert-butyl ether.

In one embodiment, isopropyl (chloro(phenoxy)phosphoryl)-Z-alaninate in the nucleophilic substitution reaction can be synthesized by reacting phenyl phosphorodichloridate with isopropyl L-alaninate as described in PCT Application WO 90/05736:

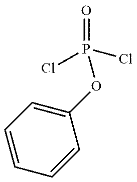 [SMILES: O=P(Cl)(Cl)Oc1ccccc1]

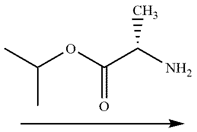

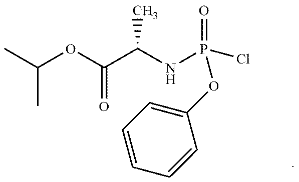

In an alternative aspect, the process for the synthesis of the diastereomerically enriched Sₚ-phosphoramidate nucleotide of Formula XVI is provided:
(a) contacting a compound of Formula I with a compound of Formula XVII in the presence of an organolithium or organomagnesium reagent and optionally in the presence of an alkali halide to afford a protected diastereomerically enriched Sₚ-phosphoramidate nucleotide of Formula XVIII:

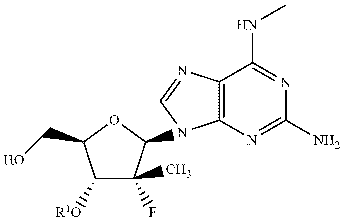

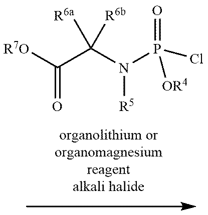

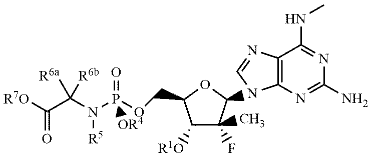

(b) further purifying the diastereomerically enriched Sₚ-phosphoramidate nucleotide of Formula XVIII to afford the diastereomerically pure Sₚ-purine phosphoramidate nucleotide of Formula XVII wherein the diastereomeric purity is greater than about 90%, greater than about 95%, or even about 99% or greater; and,
(c) deprotecting the protected phosphoramidate nucleotide of Formula XVIII to afford the diastereomerically pure Sₚ-phosphoramidate nucleotide of Formula XVI:

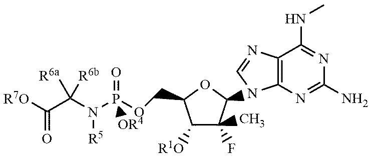

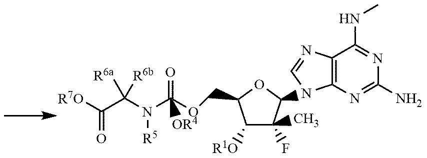

; and
(d) further optionally purifying the diastereomerically enriched compound of Formula XVI afford the diastereomerically pure compound of Formula XVI wherein the diastereomeric purity is greater than about 90%, about 95% or even about 99% or greater; and,
(e) optionally converting the compound of Formula XVI to a pharmaceutically acceptable salt of a compound of Formula XVI;

In an alternative embodiment, step (a) comprises contacting a compound of Formula I with a compound of Formula IA in the presence of an organolithium or organomagnesium reagent and optionally in the presence of an alkali halide to afford a protected diastereomerically enriched Sₚ-phosphoramidate nucleotide of Formula XVIII:

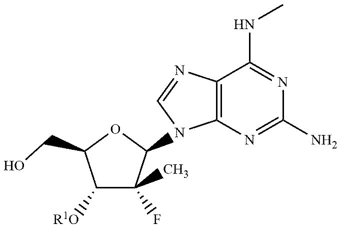

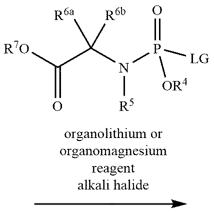

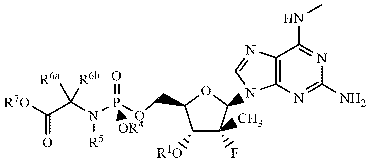

Non-limiting examples of compounds of Formula XVII include:

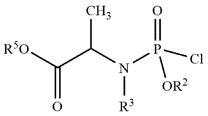

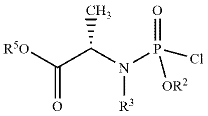

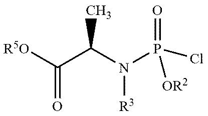

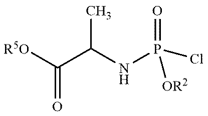

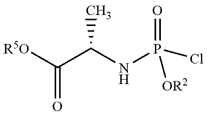

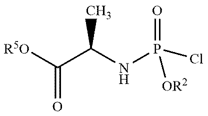

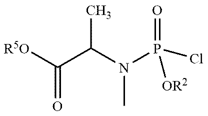

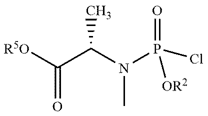

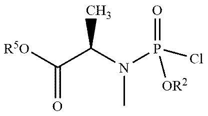

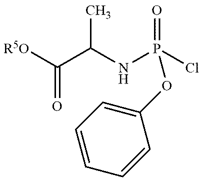

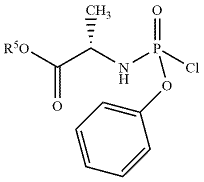

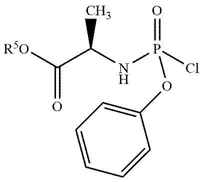

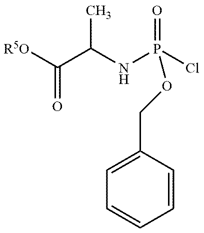

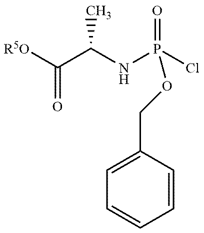

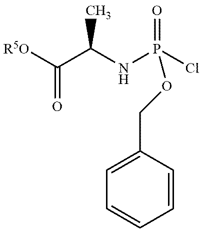

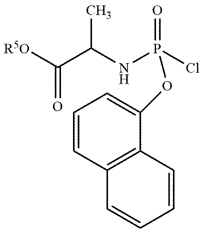

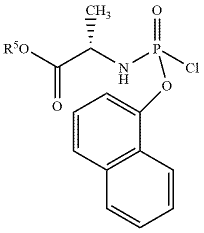

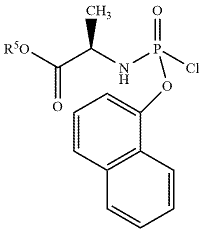

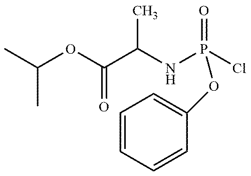 [SMILES: CC(C)OC(=O)C(C)NP(=O)(Cl)Oc1ccccc1]

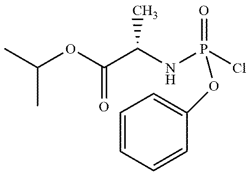 [SMILES: CC(C)OC(=O)[C@H](C)NP(=O)(Cl)Oc1ccccc1]

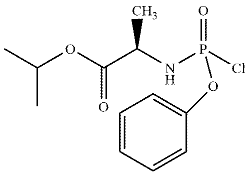 [SMILES: CC(C)OC(=O)[C@@H](C)NP(=O)(Cl)Oc1ccccc1]

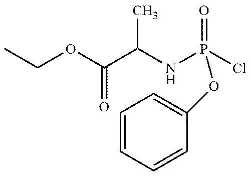 [SMILES: CCOC(=O)C(C)NP(=O)(Cl)Oc1ccccc1]

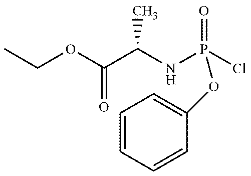 [SMILES: CCOC(=O)[C@H](C)NP(=O)(Cl)Oc1ccccc1]

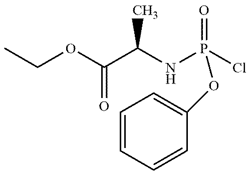 [SMILES: CCOC(=O)[C@@H](C)NP(=O)(Cl)Oc1ccccc1]

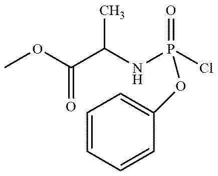 [SMILES: COC(=O)C(C)NP(=O)(Cl)Oc1ccccc1]

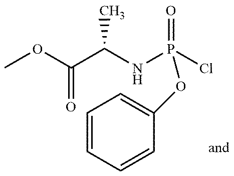

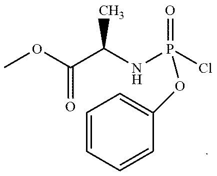

Non-limiting examples of compounds of Formula IA include:

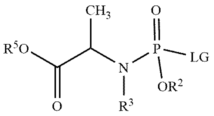

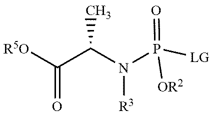

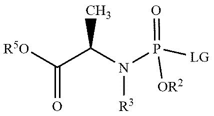

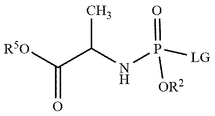

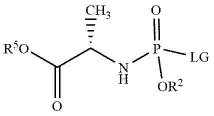

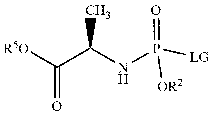

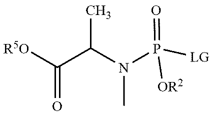

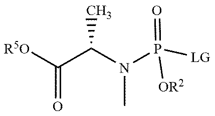

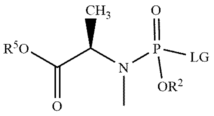

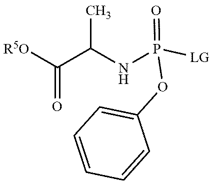

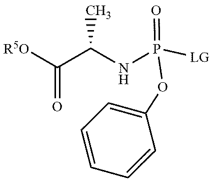

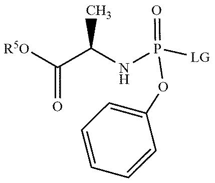

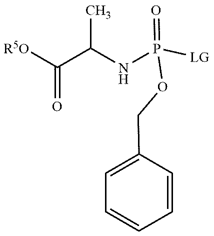

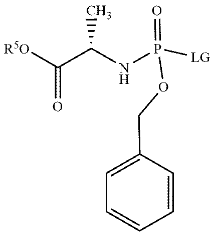

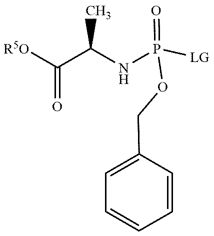

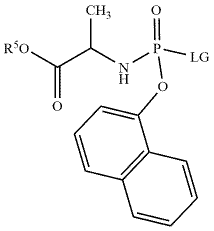

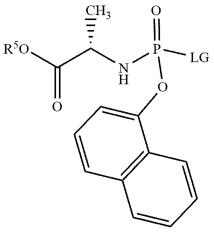

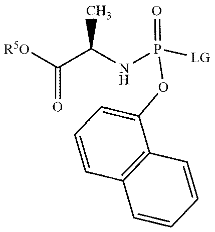

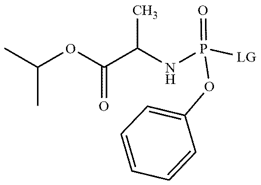

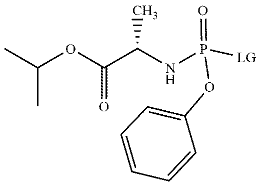

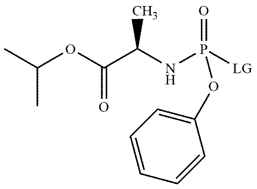

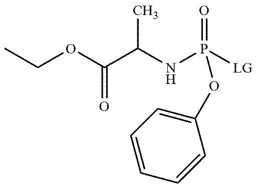

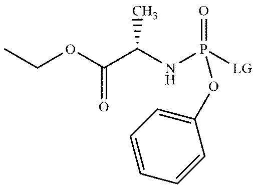

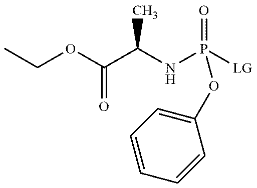

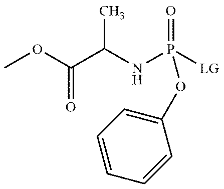

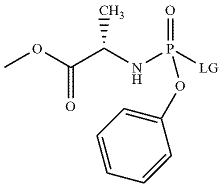

and

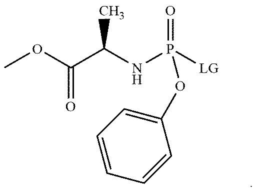

In one embodiment, LG is Br.

In one embodiment, LG is I.

In one embodiment, LG is tosylate.

In one embodiment, LG is mesylate.

In one embodiment, LG is trifluoroacetate.

In one embodiment, LG is trifluorosulfonate.

In one embodiment, LG is camphorsulfonate.

In one embodiment, LG is triflate.

In one embodiment, LG is acetate.

In one embodiment, LG is -OSO₂C₁₋₄alkyl.

In one embodiment, LG is -OSO₂aryl.

In one embodiment, LG is aryloxide.

In one embodiment, LG is aryloxide substituted with at least one electron withdrawing group

selected from halogen, NO₂, haloalkyl, —C(O)(alkyl), —C(O)(aryl), —C(O)O(alkyl), and — C(O)O(aryl).

In one embodiment, LG is aryloxide substituted with at least one halogen group.

In one embodiment, LG is aryloxide substituted with at least one NO₂ group.

In one embodiment, LG is p-nitrophenoxide.

In one embodiment, LG is 2-chlorophenoxide.

In one embodiment, LG is 4-chlorophenoxide.

In one embodiment, LG is 2,4-dinitrophenoxide.

In one embodiment, LG is pentafluorophenoxide.

In certain embodiments, the purification of the diastereomerically enriched Sₚ-phosphoramidate nucleotide of Formula II, Formula X, Formula XIII, or Formula XV to afford the corresponding diastereomerically pure Sₚ-purine phosphoramidate nucleotide is conducted via selective crystallization from an alkyl acetate, such as ethyl acetate, or a chlorinated solvent, such as dichloromethane, a ketone solvent, such as acetone, or a mixture thereof. In certain embodiments, the purification is conducted from crystallization from an alkyl acetate, chlorinated solvent, a ketone solvent, or a mixture thereof, with acetonitrile or an aliphatic hydrocarbon. In an alternative embodiment, the crystallization is conducted in isopropyl acetate.

In certain embodiments, the purification in step is the crystallization of the enriched mixture wherein the enriched mixture is dissolved in an organic solvent and then an anti-solvent is added dropwise to the above solution system wherein the organic solvent comprises, for example, a solvent selected from C₁₋₈ alcohol(s), C₂₋₈ ether(s), C₃₋₇ ketone(s), C₃₋₇ ester(s), C₁₋₂ chlorocarbon(s), and C₂₋₇ nitrile(s) and wherein the anti-solvent that is not substantially miscible with the solvent, such as C₅-₁₂ saturated hydrocarbon(s), C₆-₁₂ aromatic hydrocarbon(s), and petroleum ether. In certain embodiments, the organic solvent is selected from ethyl acetate, tert-butyl methyl ether, isopropanol or tetrahydrofuran. In an alternative embodiment, the organic solvent is isopropyl acetate. In certain embodiments, the anti-solvent is selected from petroleum ether or hexane.

In certain embodiments, the purification of the diastereomerically enriched Sₚ-phosphoramidate nucleotide Compound 1 to afford the diastereomerically pure Sₚ-purine phosphoramidate nucleotide Compound 1 is conducted via crystallization from an alkyl acetate, such as ethyl acetate, or a chlorinated solvent, such as dichloromethane, or a mixture thereof. In certain embodiments, the purification is conducted via crystallization from an alkyl acetate, chlorinated solvent, or a mixture thereof, with acetonitrile or an aliphatic hydrocarbon.

The deprotection conditions of Formula II, Formula X, Formula XIII, or Formula XV to afford Compound 1 are those generally known to the skilled artisan and are those described in Theodora W. Green, Protective Groups in Organic Synthesis, Third Edition, John Wiley & Sons (1999).

For example, when a protecting group selected from R¹, R^{3a}, and R^{3b} is independently tert-butoxycarbonyl (Boc), the protecting group(s) can be removed via conditions described on pages 281 and 520-525, including the use of HC1 in EtOAc; AcCl in MeOH; CF₃COOH in PhSH; and, TsOH in THF.

When a protecting group selected from R¹, R^{3a}, and R^{3b} is independently benzyloxycarbonyl (Cbz), the protecting group(s) can be removed via conditions described on pages 520-522, including: hydrogenation (H₂/Pd-C) and strongly acidic conditions (HBr, AcOH; 50% CF₃COOH; 70% HF, pyridine, CF₃SO₃H; FSO₃H, and CH₃SO₃H).

When a protecting group selected from R¹, R^{3a}, and R^{3b} is independently a substituted benzyl group, the protecting group(s) can be removed via conditions described on pages 86-101. For example, when R¹ is para-methoxybenzyl, deprotection conditions include DDQ (2,3-dichloro-5,6-dicyano-1,4-benzoquinone), CH₂CI₂; and catalytic DDQ (2,3-dichloro-5,6-dicyano-1,4-benzoquinone), FeCl₃, CH₂CI₂, H₂O.

When a protecting group selected from R¹, R^{3a}, and R^{3b} is independently para-methoxybenzyloxymethyl, the protecting group(s) can be removed via conditions described on page 37, including 3:1 TFH-6 M HCl.

The deprotection of the compound of Formula X, Formula XIII, or Formula XV wherein the R², the R^{3a}, and/or the R^{3b} group need to be removed are also those generally known to a skilled artisan and described in Theodora W. Green, Protective Groups in Organic Synthesis, Third Edition, John Wiley & Sons (1999). For example, the R¹ groups can be removed as discussed above and the R^{3a} and/or R^{3b} group can be removed as described in the text on pages 504-537 and 573-586. For example, when R^{3a} and/or R^{3b} is a methyl carbamate, R^{3a} and/or R^{3b} can be removed using HBr in AcOH and when R^{3a} and/or R^{3b} is a benzyl group, R^{3a} and/or R^{3b} can be removed using Pd/C in the presence of HCOOH.

Additional optional steps include:

(d) further optionally purifying the diastereomerically enriched Compound 1 to afford the diastereomerically pure Compound 1 wherein the diastereomeric purity is greater than about 90%, about 95% or even about 99% or greater; and,
(e) preparing the pharmaceutically acceptable salt form of the diastereomerically pure Sₚ-purine phosphoramidate nucleotide Compound 1.

In one embodiment, the pharmaceutically acceptable salt form of Compound 1 is the hemi-sulfate salt form, Compound 1-A:

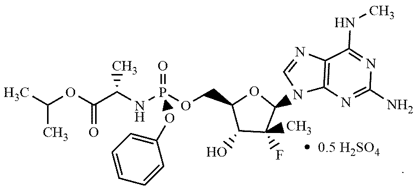

In one embodiment, Compound 1-A is prepared from Compound 1 by the dropwise addition of concentrated H₂SO₄ in MeOH and the filtration of the resulting precipitate.

The amino acid of the salt form can be in the D- or L-configuration, or a mixture thereof, including a racemic mixture. In an alternative embodiment, the pharmaceutically acceptable salt form of Compound 1-A has an alternative configuration at the amino acid:

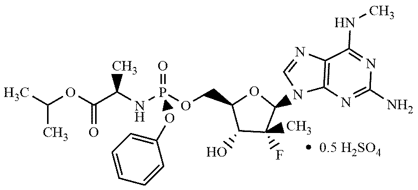

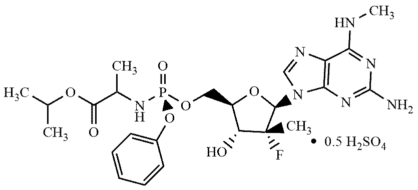

Non-limiting examples of a compound of Formula XVI synthesized by the process of the present invention include:

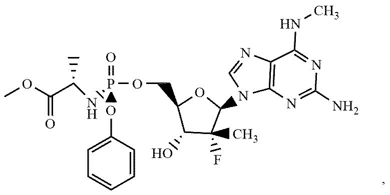

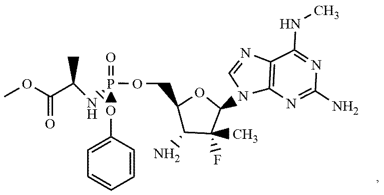

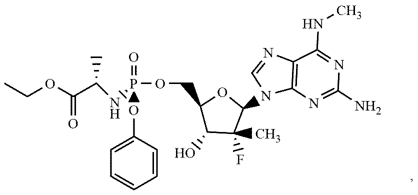

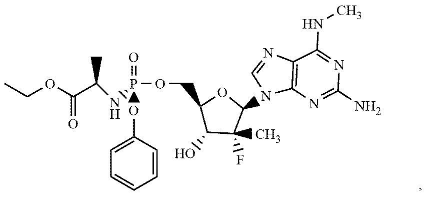

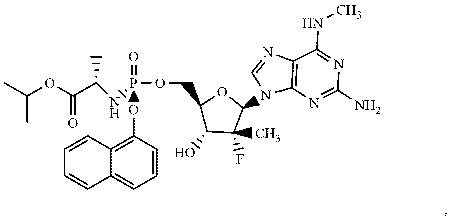

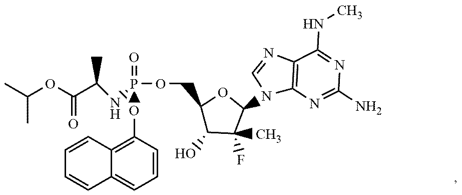

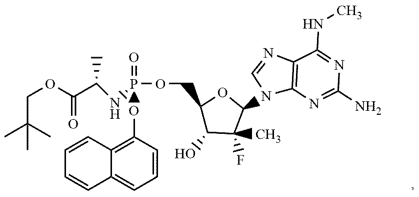

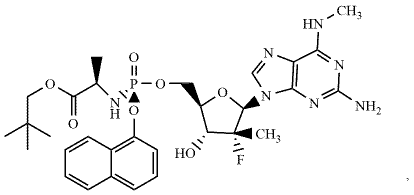

or a pharmaceutically acceptable salt thereof.

### EXAMPLES

### Example 1A- General Synthetic Scheme

Using the processes described herein, Compound 1 and Compound 1-A can be prepared. For example, a general synthetic scheme is provided below that shows the steps that can be utilized.

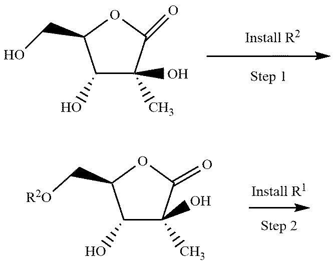

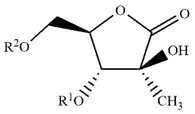

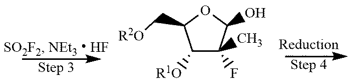

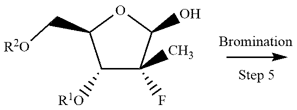

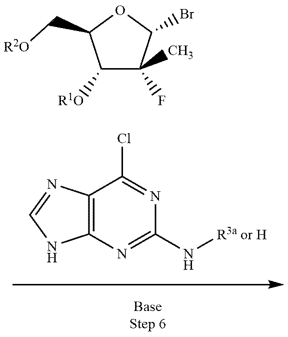

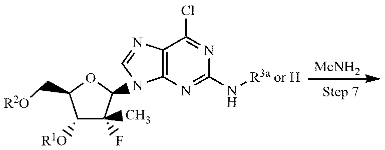

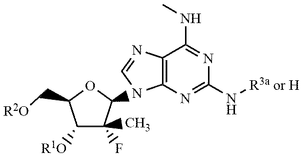

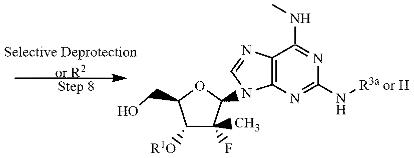

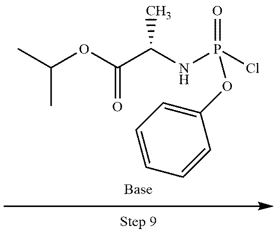

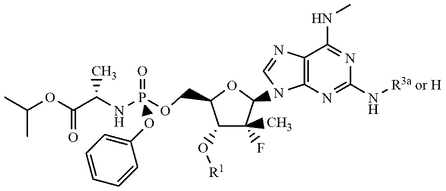

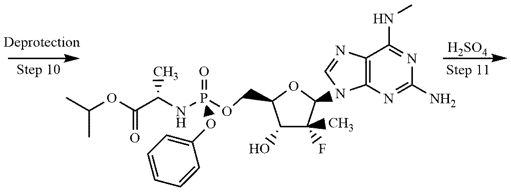

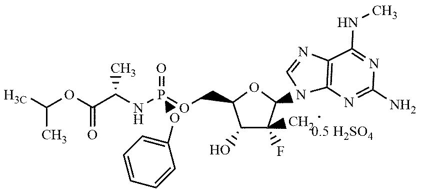

Step 1: (3S,4R,5R)-3,4-dihydroxy-5-(hydroxymethyl)-3-methyldihydrofuran-2(3H)-one is selectively protected with an R² group on the primary alcohol to afford a compound of Formula A.

Step 2: The remaining hydroxyl on the compound of Formula A is then protected with an R¹ group which is orthogonal (can be removed selectively) to the R² protecting group to afford a compound of Formula B.

Step 3: The hydroxy group is converted to fluorine with inversion of stereochemistry to afford a compound of Formula C.

Step 4: The ketone on the compound of Formula C is then reduced to afford a hydroxyl group to afford a compound of Formula D. In certain embodiments the reduction is stereoselective.

Step 5: The hydroxyl on the compound of Formula D is then displaced in a bromination reaction inverting the stereocenter to afford a compound of Formula R.

Step 6: The bromine on the compound of Formula D is then displaced by a nucleotide in a nucleophilic reaction to afford a compound of Formula F.

Step 7: The nucleotide on the compound of Formula F is then reacted with methyl amine to afford a compound of Formula G.

Step 8: The R² group on the compound of Formula G is then selectively deprotected in such a way as to maintain the R¹ group and afford a compound of Formula H.

Step 9: A phosphoramidates is then formed from the compound of Formula H to afford a compound of Formula I in a stereo-enhanced fashion.

Step 10: The compound of Formula I is then deprotected, removing the R² group and the R³ group if one is present and affording Compound 1.

Step 11: The hemi-sulfate of Compound 1 can then be formed via reaction with sulfuric acid.

### Example 1B - General Synthetic Scheme

In an alternative embodiment, the compound of Formula C can be synthesized as using the general process as described below:

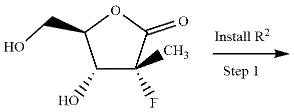

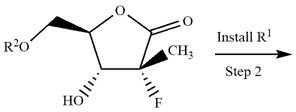

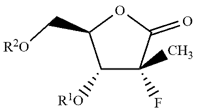

Step 1: (3R,4R,5R)-3-fluoro-4-hydroxy-5-(hydroxymethyl)-3-methyldihydrofuran-2(3H)-one is selectively protected with an R² group on the primary alcohol to afford a compound of Formula J.

Step 2: The remaining hydroxyl on the compound of Formula J is then protected with an R¹ group which is orthogonal (can be removed selectively) to the R² protecting group to afford a compound of Formula C.

### Example 2 - Non-Limiting Examples of Step 1 Protecting Groups

Selective R² protecting groups may be used to accomplish the manufacture described herein. Non-limiting examples of R² groups and examples of how to make them include:

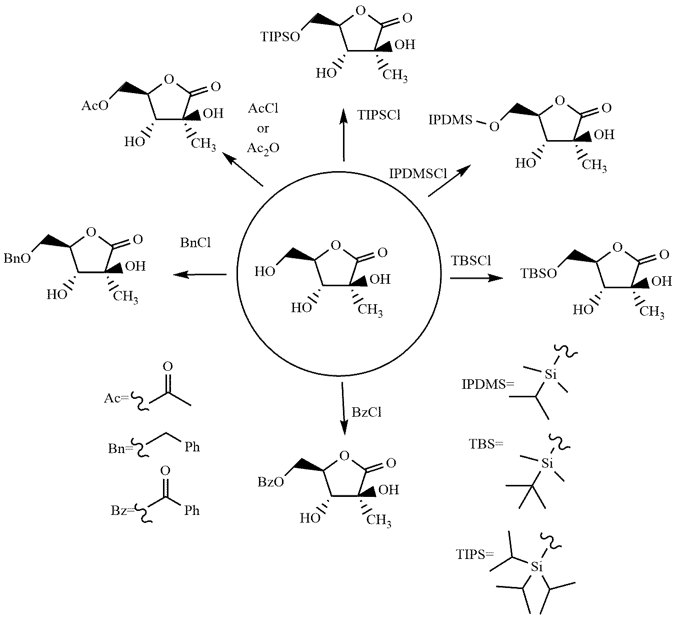

### Example 3 - Non-Limiting Examples of Step 2 Protecting Groups

Selected R¹ protecting groups are used to achieve processes described herein. These R¹ protecting groups are selected to direct stereoselectivity and to be able to be independently added or removed versus the R² protecting group. For example, if R² is a silyl protecting group then R¹ may be a non-silyl ether a bulky ester protecting group, or a bulky carbonate protecting group. If R² is a silyl protecting group then the R¹ group can also be a silyl protecting group so long as it can be removed selectively.

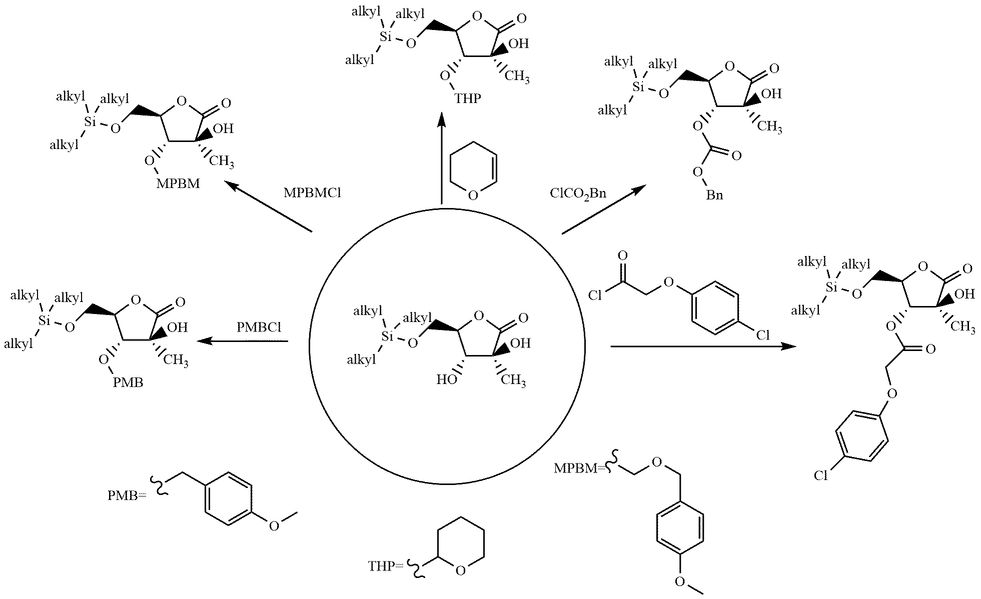

The groups listed above are not exhaustive and the skilled artisan will recognize that several other protecting groups can be used.

Non-limiting examples of nonsilyl ethers include: MPBM, NBOM, p-nitrobenzyloxymethyl ether (p-NO₂C₆H₄CH₂OCH₂OR), t-butoxymethyl ether (t-BuOCH₂OR), 2,2,2-trichloroethoxymethyl ether (CI₃CCH₂OCH₂OR), Tetrahydropyranyl ether (THP-OR), 3-bromotetrahydropyranyl ether (3-BrTHP-OR), Tetrahydrothiopyranyl ether, 1-methoxycyclohexyl ether, 1,4-dioxan-2-yl ether, Tetrahydrofuranyl ether, Tetrahydrothiofuranyl ether, p-chlorophenyl ether, p-methoxyphenyl ether, p-nitrophenyl ether, 2,4-dinitrophenyl ether, 2,3,5,6-tetrafluoro-4-(trifluoromethyl)phenyl ether, 2 or 4-picolyl ether, or 1,3-benzodithiolan-2-yl ether.

Non-limiting examples of bulky ester protecting groups include p-chlorophenoxyacetate ester (ROCOCH₂OC₆H₄-p-Cl), 3-phenylpropionate ester (ROCOCH₂CH₂Ph), and p-phenylbenzoate ester (ROCOC₆H₄-p-C₆H₄).

Non-limiting examples of bulky carbonate protecting groups include p-nitrophenyl carbonate (ROCOOC₆H₄-p-NO₂), benzyl carbonate (ROCO₂Bn), p-methoxybenzyl carbonate (p-MeOC₆H₄CH₂OCO₂R), o-nitrobenzyl carbonate (ROCO₂CH₂C₆H₄-o-NO2), and p-nitrobenzyl carbonate (ROCO₂CH₂C₆H₄-p-NO2).

### Example 4 - Representative Examples of Protecting Groups and Reagents

Non-limiting examples of protecting groups and reagents include.

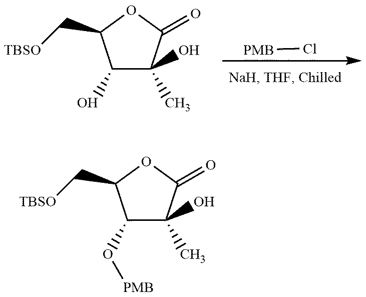

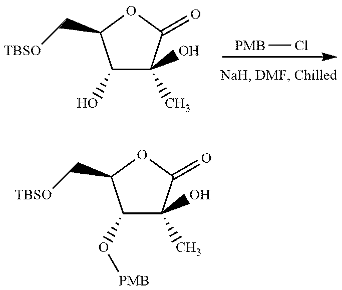

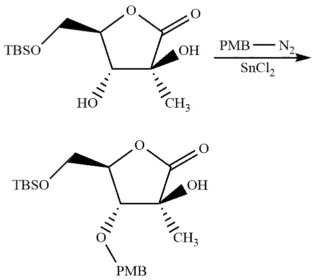

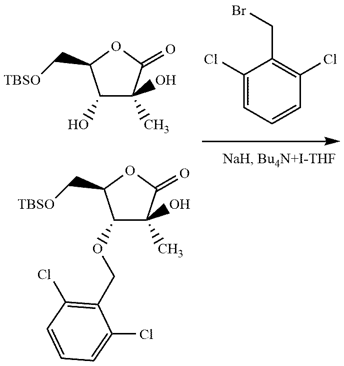

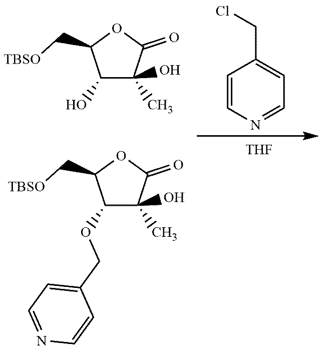 [SMILES: CC(C)(C)[Si](C)(C)OC[C@H]1OC(=O)[C@@](C)(O)[C@@H]1O ; ClCc1ccncc1 ; CC(C)(C)[Si](C)(C)OC[C@H]1OC(=O)[C@@](C)(O)[C@@H]1OCc1ccncc1 ; "THF"]

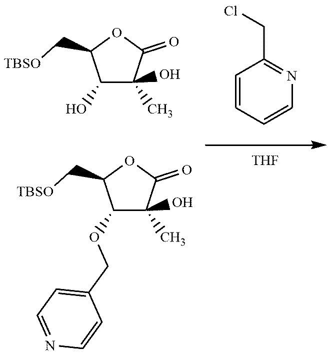 [SMILES: CC(C)(C)[Si](C)(C)OC[C@H]1OC(=O)[C@@](C)(O)[C@@H]1O ; ClCc1ccccn1 ; CC(C)(C)[Si](C)(C)OC[C@H]1OC(=O)[C@@](C)(O)[C@@H]1OCc1ccncc1 ; "THF"]

This specification has been described with reference to embodiments of the invention. However, one of ordinary skill in the art appreciates that various modifications and changes can be made without departing from the scope of the invention as set forth in the claims below. Accordingly, the specification is to be regarded in an illustrative rather than a restrictive sense, and all such modifications are intended to be included within the scope of invention.

I claim:

1. A process for preparing a 3’-protected diastereomerically enriched Sₚ-phosphoramidate nucleotide of Formula II comprising contacting a nucleoside compound of Formula I protected at the 3’-position with a protecting group which induces a stereoselective reaction with isopropyl (chloro(phenoxy)phosphoryl)-Z-alaninate in the presence of an organolithium or organomagnesium reagent in the presence of lithium chloride to afford a protected diastereomerically enriched Sₚ-phosphoramidate nucleotide of Formula II: 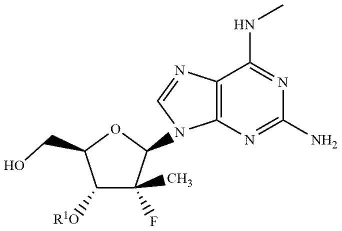 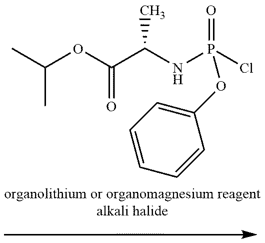 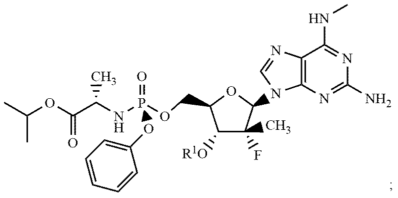
wherein:
R¹ is an oxygen protecting group which when attached to the oxygen is a moiety selected from a substituted benzyl ether, 4-bromobenzoate, p-methoxybenzyloxymethyl ether (MPBM), o-nitrobenzyloxymethyl ether (NBOM), p-nitrobenzyloxymethyl ether, t-butoxymethyl ether, 2,2,2-trichloroethoxymethyl ether, 3-bromotetrahydropyranyl ether, tetrahydropyranyl ether, tetrahydrothiopyranyl ether, 1-methoxycyclohexyl ether, 1,4-dioxan-2-yl ether, tetrahydrofuranyl ether, tetrahydrothiofuranyl ether, a substituted phenyl ether, 2-picolyl ether, 4-picolyl ether, 1,3-benzodithiolan-2-yl ether, p-chlorophenoxyacetate ester, 3-phenylpropionate ester, p-phenylbenzoate ester, alkyl p-nitrophenyl carbonyl, alkyl benzyl carbonyl, alkyl p-methoxybenzyl carbonyl, alkyl o-nitrobenzyl carbonyl, alkyl p-nitrobenzyl carbonyl, t-butylcarbonyl (Boc), and benzylcarbonyl (Cbz) and wherein the substituent is selected from alkoxy, hydroxy, nitro, bromo, chloro, fluoro, azido, and haloalkyl.

2. The process of claim 1, wherein R¹ selected from a substituted benzyl ether, p-methoxybenzyloxymethyl ether (MPBM), tetrahydropyranyl ether, tetrahydrothiopyranyl ether, t-butylcarbonyl (Boc), and benzylcarbonyl (Cbz) wherein the substituent is selected from alkoxy, hydroxy, nitro, bromo, chloro, fluoro, azido, and haloalkyl.

3. The process of claim 1, wherein R¹ is an oxygen protecting which when attached to the oxygen is a benzyl ether.

4. The process of claim 1, wherein R¹ is an oxygen protecting group which when attached to the oxygen is p-methoxybenzyl ether, 2,4-dimethoxybenzyl ether, 2-hydroxybenzyl ether, or 3,4-dimethoxybenzyl ether.

5. The process of claim 1, wherein R¹ is an oxygen protecting group which when attached to the oxygen is 4-bromobenzoate, p-methoxybenzyloxymethyl ether (MPBM), o-nitrobenzyloxymethyl ether (NBOM), p-nitrobenzyloxymethyl ether, or t-butoxymethyl ether.

6. The process of claim 1, wherein R¹ is an oxygen protecting group which when attached to the oxygen is 2,2,2-trichloroethoxymethyl ether, 3-bromotetrahydropyranyl ether, tetrahydropyranyl ether, tetrahydrothiopyranyl ether, 1-methoxycyclohexyl ether, or 1,4-dioxan-2-yl ether.

7. The process of claim 1, wherein R¹ is an oxygen protecting group which when attached to the oxygen is t-butylcarbonyl (Boc) or benzylcarbonyl (Cbz).

8. The process of claim 1, wherein the organolithium or organomagnesium reagent is an organomagnesium reagent.

9. The process of claim 8, wherein the organomagnesium is iPrMgCl.

10. The process of claim 1, wherein the organolithium or organomagnesium reagent is an organolithium reagent.

11. The process of claim 10, wherein the organolithium reagent is tert-butyl lithium.

12. The process of claim 1, wherein the ratio of Sₚ:Rₚ diastereomers in the diastereomerically enriched Sₚ-phosphoramidate nucleotide of Formula II is greater than about 70:30.

13. The process of claim 1, wherein the ratio of Sₚ:Rₚ diastereomers in the diastereomerically enriched Sₚ-phosphoramidate nucleotide of Formula II is greater than about 80:20.

14. The process of claim 1, wherein the ratio of Sₚ:Rₚ diastereomers in the diastereomerically enriched Sₚ-phosphoramidate nucleotide of Formula II is greater than about 60:40.

15. The process of claim 1, wherein the diastereomerically enriched compound of Formula II is further purified to afford diastereomerically pure S_{P}-purine phosphoramidate nucleotide, wherein the diastereomeric purity is greater than about 90%, followed by deprotection to Compound 1: 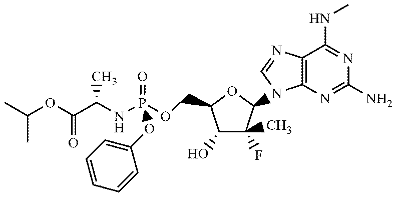 [SMILES: CNc1nc(N)nc2c1ncn2[C@@H]1O[C@H](CO[P@@](=O)(N[C@@H](C)C(=O)OC(C)C)Oc2ccccc2)[C@@H](O)[C@@]1(C)F]
.

16. The process of claim 15, wherein the further purification is selected from selective crystallization, trituration, and column chromatography.

17. A compound of Formula I: 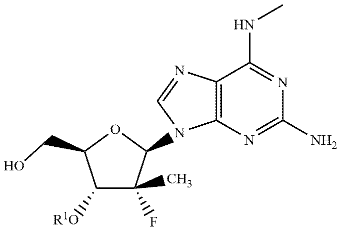
wherein R
¹ is an oxygen protecting group which when attached to the oxygen is a moiety selected from a substituted benzyl ether, 4-bromobenzoate, p-methoxybenzyloxymethyl ether (MPBM), o-nitrobenzyloxymethyl ether (NBOM), p-nitrobenzyloxymethyl ether, t-butoxymethyl ether, 2,2,2-trichloroethoxymethyl ether, 3-bromotetrahydropyranyl ether, tetrahydropyranyl ether, tetrahydrothiopyranyl ether, 1-methoxycyclohexyl ether, 1,4-dioxan-2-yl ether, tetrahydrofuranyl ether, tetrahydrothiofuranyl ether, a substituted phenyl ether, 2-picolyl ether, 4-picolyl ether, 1,3-benzodithiolan-2-yl ether, p-chlorophenoxyacetate ester, 3-phenylpropionate ester, p-phenylbenzoate ester, alkyl p-nitrophenyl carbonyl, alkyl benzyl carbonyl, alkyl p-methoxybenzyl carbonyl, alkyl o-nitrobenzyl carbonyl, alkyl p-nitrobenzyl carbonyl, t-butylcarbonyl (Boc), and benzylcarbonyl (Cbz) and wherein the substituent is selected from alkoxy, hydroxy, nitro, bromo, chloro, fluoro, azido, and haloalkyl.

18. The compound of claim 17 selected from the formula: 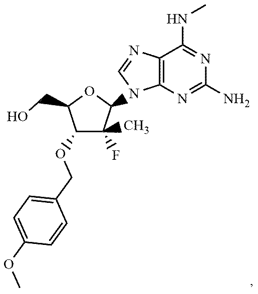 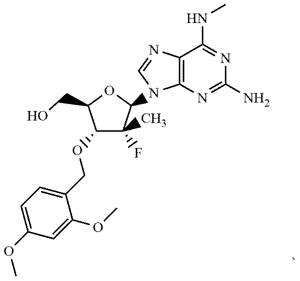 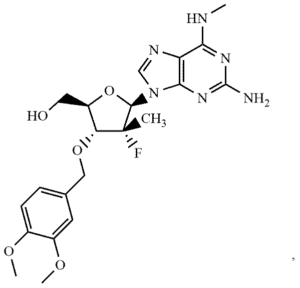 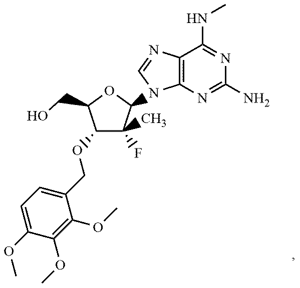 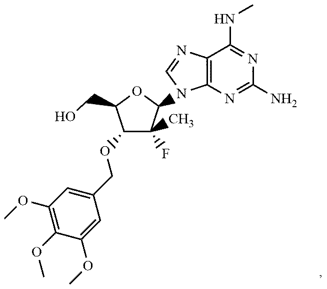
and 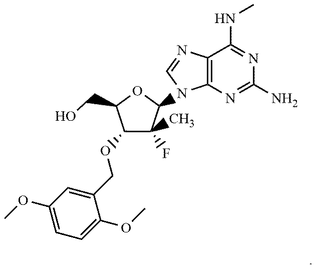
.

19. The compound of claim 17 selected from the formula: 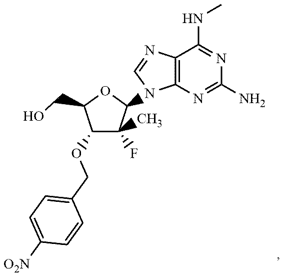 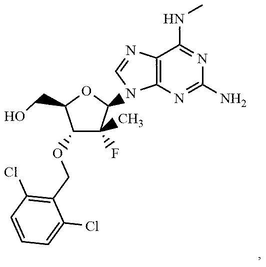 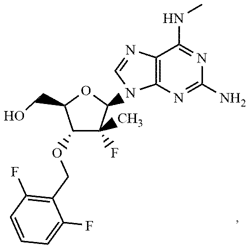
and 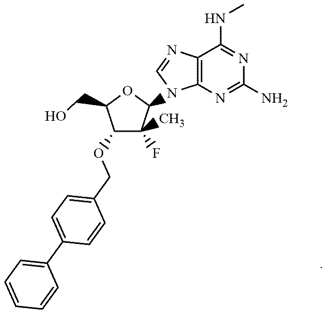
.

20. The compound of claim 17 selected from the formula: 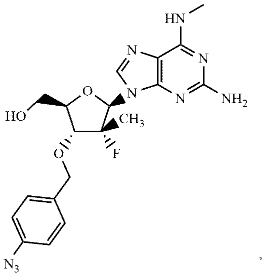 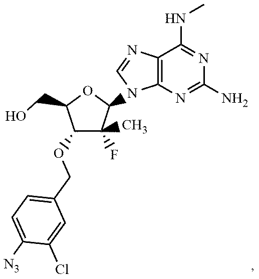
and 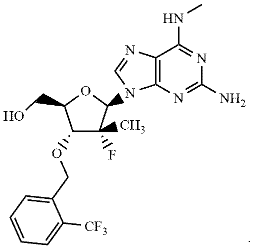
.
